(19) United States
(12) Patent Application Publication
(10) Pub. No.: US 2002/0132225 A1
(43) Pub. Date: Sep. 19, 2002

(54) Compositions and methods for prolonging survival of chilled platelets

(76) Inventors: Thomas P. Stossel, Belmont, MA (US); John H. Hartwig, Jamaica Plain, MA (US); Denisa D. Wagner, Wellesley, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/007,856

(22) Filed: Nov. 5, 2001

Related U.S. Application Data
(60) Provisional application No. 60/246,226, filed on Nov. 6, 2000.

(51) Int. Cl. C12Q 1/00; G01N 33/53; G01N 33/567

(52) U.S. Cl. 435/4; 435/7.2

(57) ABSTRACT

Compositions and methods for prolonging the survival of chilled platelets are provided. The compositions include agents which inhibit the liver macrophage binding to chilled platelets.

### RELATED APPLICATIONS

This application claims priority under 35 U.S.C. §119 to U.S. Provisional Application Serial No. 60/246,226, filed Nov. 6, 2000, entitled “Compositions and Methods for Prolonging Survival of Chilled Platelets”, the entire contents of which are incorporated herein by reference.

### GOVERNMENT RIGHTS

This invention was funded in part under National Institute of Health Grant No. HL56949. The government may retain certain rights in the invention.

### FIELD OF THE INVENTION

The inventions relate to compositions and methods for prolonging survival of chilled platelets.

### BACKGROUND OF THE INVENTION

Platelets circulate in blood as thin discs with smooth surfaces and an extensive internal membrane system called the open canalicular system. Platelets function in normal blood homeostasis by preventing excess bleeding in response to vascular injury. Exposure of sub-endothelial basement membrane or cytokines secreted by damaged endothelial cells activate platelets to change shape from their resting forms into active forms that rapidly spread and plug the vascular leak.

A reduction in the number of circulating platelets to below ˜70,000 per μL reportedly results in a prolongation of a standardized cutaneous bleeding time test, and the bleeding interval prolongs, extrapolating to near infinity as the platelet count falls to zero. Patients with platelet counts of less than 20,000 per μL are thought to be highly susceptible to spontaneous hemorrhage from mucosal surfaces, especially when the thrombocytopenia is caused by bone marrow failure and when the affected patients are ravaged with sepsis or other insults. The platelet deficiencies associated with bone marrow disorders such as aplastic anemia, acute and chronic leukemias, metastatic cancer but especially resulting from cancer treatment with ionizing radiation and chemotherapy represent a major public health problem. Thrombocytopenia associated with major surgery, injury and sepsis also eventuates in administration of significant numbers of platelet transfusions.

The standard therapeutic response to the thrombocytopenia syndromes summarized above is allogeneic platelet transfusion. The technology for platelet transfusion used today was in large measure developed over 20 years ago. One of the principal elements of platelet transfusion procedure is based on the startling fact that in contrast to storage of nearly every other biological product, platelets must be kept at room temperature. This practice resulted from evidence that refrigerated platelets had a much shorter survival (one day or less) and consequently much poorer hemostatic effectiveness in thrombocytopenic or aspirin-treated recipients than fresh platelets kept at room temperature which could circulate for up to a week. This observation was consonant with the observation that chilling of platelets below about 15° C. results in conversion of the discoid shape of the freshly drawn platelet to a distorted spiny object. Rewarming of the cooled platelets, first thought to reverse the shape change, actually resulted in spheres rather than discs, and extensive analyses of platelet functions in the 1970s established that retention of a discoid shape was one of the best indicators of a hemostatically viable platelet for transfusion purposes. More recent work has shown that cooling and rewarming platelets also causes a rise in intracellular calcium.

Adjustments to the requirement for room temperature storage of platelets procured for transfusion have included development of gas-porous bags with a large surface area. Agitation of these bags is used to maximize diffusion of CO₂ that accumulates in the platelet concentrates which continue to metabolize at room temperature. Without the suppression of energy metabolism at refrigeration temperatures, CO₂ accumulates and lowers the ambient pH, and acid is highly deleterious to platelet function. As a result, one problem with room temperature storage of platelets is the need for special equipment and temperature controls separate from those used for red blood cell preservation. Another consequence of room temperature storage is a widely recognized “storage lesion” characterized by a rapid diminution in in vitro tests of platelet function believed important for hemostatic effectiveness in vivo. While the causes of functional deterioration during room temperature storage are not definitely known, possible reasons include mechanical effects of agitation, toxic metabolite buildup despite preventive measures, and the greater activity of platelet- and white blood cell-derived proteases at room temperature compared to the cold and elaboration of toxic leukocyte oxidants. Leukocyte-platelet interactions during room-temperature storage also lead to the release of cytokines which may contribute to post-transfusion febrile reactions. The major complication of room temperature storage, however, is the risk of bacterial growth which has resulted in the FDA limiting storage time to from 7 days to 5 days.

The processing of blood components is a highly regulated activity. Platelet concentrates must be subjected to eight laboratory tests, and this testing and the associated quality control requirements surrounding the testing procedures in effect reduce the storage time to three days. The FDA-mandated decrease in storage time notably increased the fraction of platelets that became outdated, and outdating represents a significant cost to the blood procurement industry.

As of four years ago, the estimated annual utilization of platelets in the USA was ˜7 million platelet concentrates from single whole blood donations stored for a mean duration of 4.5 days and 300,000 concentrates from single donor pheresis procedures utilized more efficiently with a mean storage time of 1.5 days. The frequency of platelet transfusion had increased at a constant rate of about 10% annually since 1980. The increase in demand for platelet transfusions is a result primarily of increasingly aggressive cytoreductive therapy for neoplastic disease. The development of thrombopoietin, should it sustain platelet production in myelosuppressed patients, is just as likely to promote more aggressive myeloablative therapy as followed the introduction of G-CSF and other myeloid growth factors, so that requirements for platelet transfusion support would not necessarily fall. More likely to reduce demand is the fact that hematologists are currently questioned the so-called 20,000 “trigger” level in the platelet count that leads to ordering of platelet transfusions. Recently compiled data for US platelet utilization in 1994 points to a trend toward reduced demand. Although single donor apheresis collections and transfusions increased by 26% from 1992, this increase was offset by a diminution in concentrates transfused, such that total “units” transfused decreased by 5% compared with 1992.

The blood procurement system attempts to balance supply and demand. If supply exceeds demand, outdating results in wastage. Currently the industry accepts a wastage rate up to 20%, but if the wastage, under current storage constraints, falls below 2-3%, the risk of shortage becomes great, since the variation in demand exceeds several standard deviations beyond a normal distribution. In particular, weekends and holidays present a problem, because donations fall off sharply during those times. The instant invention buffers shifts in supply and demand by effectively extending platelet shelf-life. In addition, regional blood centers transship blood products to outlying users, usually by truck. The short storage time of platelets require shipments to take place at least every three days. Extension of shelf-life permitted by the instant invention would, therefore, diminish transportation costs.

The average price of a single donor platelet concentrate is currently around $500 and of a pheresis concentration of $1000. The actual cost of the bags that are used to house platelets is on the order of $3-$4 for individual (random donors), $10 for apheresis bags. The large markups are for labor and software used in platelet procurement. To put a dollar value on wastage costs, one conservative estimate would be that if there is a 10 per cent wastage rate affecting 10 million platelet procurements, where the average price per product is $200, the costs is $200 million. This figure provides a significant window for pricing a product that could reduce this wastage. In addition, even if demand for platelets should fall, particularly if it in part would result from an improved product, the $2 to $3 billion in platelets sold presents an inviting cash flow for a provider able to gain market share that might generate cost savings of this considerable sum affecting the end users, the hospitals that purchase platelets for transfusion. Taking the 1994 data on US platelet transfusion, 820,000 pheresis collections at a cost of $1000 each plus 4,120,000 concentrates collected (assuming a 15% wastage rate and 3,582,000 transfusions) at $500 each equals $2.88 billion. A 15% wastage rate then cost the system $432 million in 1994.

While efficacy of platelet transfusion is optimally determined by cessation of bleeding, this parameter is difficult to monitor in practice. Therefore, the operational definition of a successful transfusion is an arbitrary minimal increase in the circulating platelet count 10-60 minutes after the transfusion. The cutoff value is an increment of at least 30-36×10⁹ platelets per liter of blood following infusion of six units of random donor platelets or one unit of pheresis platelets into a 75 Kg recipient. Lesser increments are said to reflect refractoriness to platelet transfusion. The causes of refractoriness are numerous and include immunologic sensitization to platelet antigens, increased platelet clearance due to sepsis and hemorrhage, but arguably a major cause is the poor condition of infused platelets per se. The reality is that most transfused platelets are functionally far poorer than the fresh platelets shown to circulate for many days. The dynamics of platelet procurement, testing and short storage force blood suppliers to minimize wastage by releasing the oldest platelets first, insuring that most transfusions represent the most deteriorated platelets. Most platelet transfusions are given for bleeding risk, not actual bleeding. Therefore the more native a platelet, i.e., less likely to be toxic, the more desirable.

Although claims for hemostatic effects of frozen platelets have been reported even in recent years since the refrigeration lesion was recognized, and some research continues on cryopreservation methodology it is hard to understand how long-term platelet storage by standard cell freezing methods could be viable as long as simply cooling platelets produces a reproducibly defective product. The irreversible cold-induced shape change occurs very rapidly, much faster than the time required to bring platelets to freezing temperatures, and there is no evidence that cryopreservatives prevent this shape change. In fact, research has documented functional changes of transfusion responses in frozen and thawed platelets very similar to those observed in platelets simply refrigerated. Overcoming the refrigeration-induced storage lesion would permit platelet freezing.

Most transfusion physicians indicate that if platelets could be stored in the cold and retain hemostatic function, they would be preferable to the product currently in use. However, little if any information is known regarding the mechanism of platelet clearance, in general, and chilled platelet clearance in particular. Accordingly, a need exists to better understand the processes by which platelets are cleared in order to develop compositions and methods that are useful for addressing the above-noted problems in the platelet transfusion industry.

### SUMMARY OF THE INVENTION

The invention is based, in part, on the discovery that chilled, apoptotic and senescent platelets are cleared by distinct mechanisms. Prior to this discovery, it was not known that chilled platelets are rapidly cleared and deposited in the liver. The results disclosed herein demonstrate that liver macrophages (e.g., Kuppfer cells) are important in the clearance of chilled or apoptotic platelets and that splenic macrophages are important in the clearance of senescent platelets. Accordingly, the invention provides compositions and methods for selecting agents which are useful for prolonging the survival of platelets and using such agents to treat chilled platelets to prolong their survival.

As used herein, chilled platelets refer to platelets which have been stored at, or exposed to, a temperature less than about 22 degree C., preferably, less than about 15 degree C. and, more preferably from about 0 degree C. to about 14 degree C. As used herein, chilled platelets also embraces platelets which have been stored at or exposed to a temperature sufficient to freeze the platelets. In general, the chilled platelets have been stored at, or exposed to, the reduced temperature conditions for a time period that would have been sufficient (unless treated as discussed below) to induce shape changes characteristic of cold-activated platelets.

Methods and compositions for treating chilled platelets with retention of discoid shape are described in U.S. Pat. Nos. 5,876,676; 5,576,213; and 5,358,844; the entire contents of which are incorporated herein by reference. Exemplary agents include a first agent for inhibiting actin filament severing (e.g., an intracellular calcium chelator such as Quin-1) and a second agent for inhibiting actin polymerization (e.g., a cytochalasin) (as defined in the above-noted patents).

According to a first aspect of the invention, a first method for identifying a “platelet clearance antagonist” is provided. The method involves contacting a chilled platelet with a liver macrophage (e.g., Kuppfer cell) in the presence and in the absence of a test molecule (e.g., a molecular library); and detecting binding of the chilled platelet to the liver macrophage, wherein a reduction in the binding in the presence of the test molecule relative to the binding in the absence of the test molecule indicates that the test molecule is a platelet clearance antagonist.

As used herein, a “platelet clearance antagonist” refers to an agent which: (1) binds to a platelet ligand or binds to a liver macrophage receptor; and (2) prevents binding of the platelet ligand to the liver macrophage receptor. As used herein, platelet clearance antagonists which bind to platelet ligands are referred to as “platelet antagonists”; platelet clearance antagonists which bind to liver macrophage receptors are referred to as “receptor antagonists”. Exemplary platelet antagonists and receptor antagonists bind to the platelet ligands and liver macrophage receptors, respectively, provided in Table 1. Although not wishing to be bound to any particular theory or mechanism, it is believed that the binding of the platelet ligand to the liver macrophage receptor is involved in the differential clearance of chilled platelets compared to senescent platelets. Accordingly, platelet clearance antagonists are useful for prolonging the survival of chilled platelets in vivo.

TABLE 1
PLATELET LIGANDS/KUPPFER CELL RECEPTORS

| Platelet Ligands | Liver Macrophage Receptors: |
|---|---|
| vWfR (including GPIb α, GPIb β, | CRs (C3b, C3bi) |
| GPIX and GPV) | CR1 |
| | CR2 |
| | CR3 (αMβ2) (alternatively |
| | referred to as CD11b/CD18 or |
| | Mac-1) |
| | CR4 (αXβ2) |
| IgG bound to platelet FcRIIA | FcR family |
| | Fe gamma RI |
| | Fe gamma RIIA |
| | Fe gamma RIII (CD16) |
| PS (phosphatidyl serine) | Class B Scavenger CD36 |
| PECAM-1 | PECAM-1 |
| PECAM-1 | Vitronectin (αvβ3) |
| CD47 | SIRP α |

According to still another aspect of the invention, a second method for identifying a platelet clearance antagonist is provided. The method involves contacting an isolated platelet ligand with a liver macrophage (e.g., Kuppfer cell) in the presence and in the absence of a test molecule (e.g., library molecule(s), antibodies, etc.); and detecting binding of the platelet ligand to the liver macrophage, wherein a reduction in the binding in the presence of the test molecule relative to the binding in the absence of the test molecule indicates that the test molecule is a platelet clearance antagonist.

According to yet another aspect of the invention, a third method for identifying a platelet clearance antagonist is provided. The method involves contacting an isolated platelet ligand with an isolated liver macrophage (e.g., Kuppfer cell) receptor in the presence and in the absence of a test molecule; and detecting binding of the platelet ligand to the liver macrophage receptor, wherein a reduction in the binding in the presence of the test molecule relative to the binding in the absence of the test molecule indicates that the test molecule is a platelet clearance antagonist.

According to a still further aspect of the invention, a fourth method for identifying a platelet clearance antagonist is provided. The method involves contacting a chilled platelet with an isolated liver macrophage (e.g., Kuppfer cell) receptor in the presence and in the absence of a test molecule; and detecting binding of the chilled platelet with the liver cell receptor, wherein a reduction in the binding in the presence of the test molecule relative to the binding in the absence of the test molecule indicates that the test molecule is a platelet clearance antagonist.

According to another aspect of the invention, a method for preparing platelets for transfusion is provided. The method involves contacting a chilled platelet with a platelet antagonist under conditions to permit the chilled platelet antagonist to bind to a ligand on the chilled platelet. The contacting can be performed before, during or after chilling of the platelets; optionally, contacting can be performed while the platelets are contained in a platelet bag.

In certain embodiments, the platelet antagonist selectively binds to a platelet ligand identified in Table 1. More preferably, the platelet antagonist selectively binds to the platelet ligand that is vWfR or a subunit thereof (GPIb α, GPIb β, GPIX and GPV). Most preferably, the platelet antagonist selectively binds to GPIb α. The platelet antagonist can be any type of binding molecule, e.g., an antibody or fragment thereof, provided that the platelet antagonist selectively binds to the platelet ligand and inhibits binding of a chilled platelet to a Kuppfer cell.

According to another aspect of the invention, a composition containing one or more platelet clearance antagonists as described herein and, optionally, a plurality of platelets; is provided. The platelet clearance antagonists can include a platelet antagonist and/or a liver macrophage (e.g., Kuppfer cell) receptor antagonist. The preferred platelet antagonists selectively bind to vWfR or a subunit thereof. The preferred liver macrophage receptor antagonists selectively bind to αMβ2. Optionally, the composition further includes a pharmaceutically acceptable carrier. In these and other embodiments, the composition may be contained in a platelet bag. In a related aspect, a method for forming a medicament is provided. The method involves placing one or more platelet clearance antagonists and, optionally, a plurality of platelets in a pharmaceutically acceptable carrier.

According to one aspect of the invention, a method for increasing platelet circulatory time is provided. The method involves administering to a subject in need of such treatment, one or more platelet clearance antagonists in an amount effective to increase platelet circulatory time in the subject. Optionally, the composition further includes a plurality of chilled platelets. Additionally or alternatively, platelet antagonist-treated platelets (i.e., platelets which have been treated with one or more platelet antagonists) can be administered to a subject. Optionally, unbound platelet antagonists are removed from the composition prior to administration of the platelet antagonist-treated platelets.

According to yet another aspect of the invention, a method for treating a subject in need of platelets is provided. The method involves administering to the subject, a composition comprising: (1) a first composition containing: (a) a plurality of chilled platelets; and one or more platelet clearance antagonists; (2) a second composition containing: a plurality of platelet-antagonist-treated platelets; or (3) a third composition containing: a plurality of platelet lesion cleavage agent-treated platelets (described below), wherein the first composition or the second composition or the third composition are administered in an amount effective to treat the subject. The preferred platelet clearance antagonists are as described above. Optionally, unbound platelet antagonists or cleavage agents are removed from the composition prior to administration of the platelet antagonist-treated platelets. In these and other embodiments, the composition may further include one or more liver macrophage receptor antagonists. Chilled platelets may be contained in the composition or separately administered to the subject. In these and other embodiments, the platelets can be contained in a platelet bag to facilitate administration to the subject.

According to yet another embodiment, a method for identifying a platelet lesion cleavage agent is provided. The method involves contacting a chilled platelet with a liver macrophage or receptor thereof in the presence and in the absence of a test cleavage agent; and detecting binding of the chilled platelet to the liver macrophage, wherein a decrease in the binding in the presence of the test cleavage agent relative to the binding in the absence of the test cleavage agent indicates that the test molecule is a platelet lesion cleavage agent. Preferably, the test cleavage agent is selected from the group consisting of enzymes that cleave carbohydrates (e.g., a galactosidase, a glucosidase, a mannosidase) or that cleave proteins.

Although binding of the chilled platelet to the liver macrophage can be detected in accordance with any standard method known to one of ordinary skill in the art, a preferred method for detecting binding involves detecting phagocytosis of the chilled platelet by the liver macrophage. Although not wishing to be bound to any particular theory or mechanism, it is believed that chilling of platelets induces changes in the surface expression of platelet proteins such as vWfR or a subunit thereof, which play a role liver macrophage clearance. Accordingly, it is believed that the cleavage of such aberrant surface proteins removes surface platelet ligands that are essential for liver macrophage receptor recognition. Thus, contacting the platelet lesion cleavage agent with a chilled platelet permits the platelet lesion cleavage agent to cleave a platelet lesion on a chilled platelet and, thereby, inhibit binding of the platelet to the liver macrophage. The invention further embraces platelets that are prepared in accordance with this method.

These and other aspects of the invention will be more apparent in reference to the following detailed description of the invention.

### BRIEF DESCRIPTION OF THE FIGURES

It is to be understood that the figures are provided for illustrative purposes only and are not required for understanding or practicing the invention.

FIG. 1 shows cold-mediated platelet actin remodeling.

FIG. 2 shows the clearance rates for In¹¹¹-labeled platelets in baboons.

FIG. 3 shows that chilled platelets are rapidly removed from the circulation in mice.

FIG. 4 shows clearance sites for ¹¹¹n-labeled platelets in mice.

FIG. 5 shows a change in GPIb induced by cooling (indicated by an asterisk*).

FIG. 6 shows chilled platelets do not show the increased adherence to hepatic αMβ2 integrin knockout macrophages seen in wildtype macrophages.

FIG. 7 shows development of inhibitors to diminish the affinity of the GPb-αMβ2 integrin interaction should permit chilled and rewarmed platelets to circulate normally.

FIG. 8 shows an alternative approach involving removal of part of GPIb from platelets ex vivo, so as to render platelets that do not bind αMβ2 integrins after chilling but retain hemostatic capability.

FIG. 9 shows that chilled wildtype mouse platelets circulate with the same half-life time as platelets stored at room temperature (A) in αMβ2-integrin deficient animals (B), but not in C3-deficient mice (C).

FIG. 10 shows that αMβ2-integrin deficient liver-phagocytes also fail to bind chilled platelets; ratio of chilled to warm platelets adhering to hepatic sinusoids shown.

FIG. 11 shows the concentration dependent inhibition of CM-Orange pellet phagocytosis by stimulated THP-1 cells using the monosaccharide α-methyl-glucoside.

FIG. 12 shows the concentration dependent inhibition of CM-Orange pellet phagocytosis by stimulated THP-1 cells using the monosaccharide α-methyl-mannoside.

FIG. 13 shows the concentration dependent inhibition of CM-Orange pellet phagocytosis by stimulated THP-1 cells using the monosaccharide β-methyl-glucoside.

FIG. 14 shows the epitope map of monoclonal antibodies to GPIbα, schematically represented on the extracellular domain of GPIbα.

### DETAILED DESCRIPTION OF THE INVENTION

The ability to store platelets in the cold permits a more efficient collection of platelets before elective procedures and encourages use of single donor apheresed platelets. It also increases the availability of platelet concentrates for use on short notice and, thereby, reduces wastage and associated costs. Accordingly, the instant invention facilitates the use of chilled platelets by abrogating platelet storage lesions, i.e., cold temperature-induced changes in the expression of platelet surface ligands which mediate liver macrophage clearance, which result from platelet storage at or exposure to cold temperatures.

The invention is based, in part, on the discovery that chilled, apoptotic, and senescent platelets are cleared by distinct mechanisms. Prior to this discovery, platelet clearance mechanisms were poorly understood. To further define the mechanisms of platelet clearance, we examined the survival of fluorescently- or 111n-labeled platelets in syngeneic mice and determined the sites of deposition of labeled platelets disappearing from the circulation. Platelets kept at room temperature had a 50% recovery and a T1/2 of 42 hours. The bulk of these platelets progressively accumulated in the spleen. Ninety percent of the platelets subjected to ultraviolet radiation which induces apoptosis detectable by gelsolin degradation and annexin binding to exposed surface phosphatidylserine cleared within one hour, the liver being the site of clearance. Platelets chilled to 4 degrees C. for 1 hour also cleared rapidly and deposited in the liver; however, these platelets did not exhibit evidence of apoptosis. Both irradiated and chilled platelets associated with hepatic αMβ2-expressing cells by FACS analysis. These results implicate liver macrophages (e.g., Kuppfer cells) as important in the clearance of chilled or apoptotic platelets and splenic macrophages as important in the clearance of senescent platelets. Based, in part, on the foregoing surprising and unexpected discoveries, we disclose herein compositions and methods for selecting agents which are useful for prolonging the survival of platelets exposed to cold temperatures and using such agents to treat chilled platelets to prolong their survival.

As used herein, a subject in need of treatment refers to a mammal, preferably a human, who presently is in need of or, in future, may be in need of a platelet transfusion. One subclass of subjects are those who may be undergoing surgery, about to undergo surgery, or recently undergone surgery or other operation, injury, or treatment (e.g., chemotherapy) necessitating platelet infusion. Optionally, the subject may be presenting symptoms of a thrombocytopenia syndrome. Thrombocytopenia associated with major surgery, injury and sepsis also eventuates in administration of significant numbers of platelet transfusions. A subject having a thrombocytopenia syndrome is a subject with at least one identifiable sign, symptom, or laboratory finding sufficient to make a diagnosis of a thrombocytopenia syndrome in accordance with clinical standards known in the art for identifying such disorders. Examples of such clinical standards can be found in Harrison's Principles of Internal Medicine, 14th Ed., Fauci AS et al., eds., McGraw-Hill, New York, 1998. Additional subjects include those in need of platelet transfusions as a result of platelet deficiencies associated with bone marrow disorders such as aplastic anemia, acute and chronic leukemias, metastatic cancer but especially resulting from cancer treatment with ionizing radiation and chemotherapy.

The phrase “therapeutically effective amount” means that amount of a compound which prevents the onset of, alleviates the symptoms of, or stops the progression of a disorder or disease being treated. The phrase “therapeutically effective amount” means, with respect to a thrombocytopenia syndrome, that amount of a platelet clearance antagonist and/or platelet antagonist-treated platelets which prevents the onset of, alleviates the symptoms of, or stops the progression of the thrombocytopenia syndrome.

The term “treating” is defined as administering, to a subject, a therapeutically effective amount of a platelet clearance antagonist of the invention and/or platelet antagonist-treated platelets that is sufficient to prevent the onset of, alleviate the symptoms of, or stop the progression of a disorder or disease being treated. In a preferred embodiment, the subject is a human.

The pharmaceutical preparations disclosed herein are prepared in accordance with standard procedures and are administered at dosages that are selected to reduce, prevent or eliminate the condition (See, e.g., Remington's Pharmaceutical Sciences, Mack Publishing Company, Easton, Pa., and Goodman and Gilman's The Pharmaceutical Basis of Therapeutics, Pergamon Press, New York, N.Y., the contents of which are incorporated herein by reference, for a general description of the methods for administering various agents and platelets for human therapy).

As used herein, chilled platelets refer to platelets which have been stored at or exposed to a temperature less than about 22 degree C. The platelets are collected from peripheral blood by standard techniques known to those of ordinary skill in the art. In a preferred embodiment, the temperature is less than about 15 degree C. In a most preferred embodiment, the temperature ranges from about 0 degree C. to about 14 degree C., inclusive, and, more preferably, between about 4 degree C. to about 14 degree C., inclusive.

In general, the chilled platelets are stored at, or exposed to, a reduced temperature conditions for a time period that would have been sufficient (unless treated as discussed below) to induce shape changes characteristic of cold-activated platelets. This time period can range from minutes to hours (from about 1 hour to 23 hours, inclusive, and every hour therebetween) to days (from about a day to 30 days, inclusive, and every day therebetween), or months (from about 1 month to 1 year, inclusive).

The present invention is directed to compositions and methods employing preparations of chilled platelets. Such chilled plated preparations may or may not be treated with the agents for retaining discoid shape such as those disclosed in the above-mentioned U.S. patents. Accordingly, a brief description of cold-induced platelet activation and the agents for inhibiting same are provided below as background information for the instant invention.

As used herein, the phrase “cold-induced platelet activation” refers to the molecular and morphological changes that blood platelets undergo following exposure to cold temperatures, e.g., 4 degree C. The compositions containing the platelets of the invention typically are warmed to body temperature prior to administration to the subject. Optionally, the chilled platelets are treated with agents for inhibiting cold-induced platelet activation and preserving discoid shape prior to treatment with a platelet antagonist. Exemplary agents that are useful for inhibiting cold-induced platelet activation are discussed in more detail below. Such inhibitory agents include “first agents for inhibiting actin filament severing” and “second agents for inhibiting actin polymerization” and are the subject of U.S. Pat. Nos. 5,876,676; 5,576,213; and 5,358,844. The agents for inhibiting cold induced platelet activation may be contacted with the platelets simultaneously or sequentially. Preferably, one or more of these first and second agents is contacted with the platelets at a temperature from about room temperature to about 37 degree C. and, following treatment, the platelets are chilled to a reduced temperature as discussed above.

Cold-induced platelet activation is manifested by changes in platelet morphology, some of which are similar to the changes that result following platelet activation by, for example, contact with glass. The structural changes indicative of cold-induced platelet activation are most easily identified using techniques such as light or electron microscopy. On a molecular level, cold-induced platelet activation results in actin bundle formation and a subsequent increase in the concentration of intracellular calcium. Actin-bundle formation is detected using, for example, electron microscopy. An increase in intracellular calcium concentration is determined, for example, by employing fluorescent intracellular calcium chelators. Many of chelators for inhibiting actin filament severing are also useful for determining the concentration of intracellular calcium. Cold-activated platelets also have a characteristically reduced hemostatic activity in comparison with platelets that have not been exposed to cold temperatures. These differences in hemostatic activity are reflected in differences in actin polymerization activity. Accordingly, various techniques are available to determine whether or not platelets have experienced cold-induced activation. As discussed in U.S. patent Nos. U.S. Pat. Nos. 5,876,676; 5,576,213; and 5,358,844; (the entire contents of which are incorporated herein by reference), such techniques can be used to select the concentrations of first and second agents for inhibiting cold-induced platelet activation.

As used herein, “actin filament severing” refers to the disruption of the non-covalent bonds between subunits comprising actin filaments. Actin filament severing in the platelet, presumably by gelsolin, requires an increase in the intracellular concentration of free calcium. Accordingly, in a preferred embodiment, the first agent for inhibiting actin filament severing is an intracellular calcium chelator. Exemplary intracellular calcium chelators include the lipophillic esters (e.g., acetoxymethyl esters) of the BAPTA family of calcium chelators, e.g., QUIN, STIL, FURA, MATA, INDO, and derivatives thereof. See U.S. Pat. Nos. 5,876,676; 5,576,213; and 5,358,844 for a further discussion of these intracellular chelators.

BAPTA is an acronym for 1,2-bis(2-aminophenoxy) ethane N,N,-N′,N′-tetraacetic acid. BAPTA and “BAPTA-like” compounds share a high selectivity for calcium over magnesium. As used herein, “BAPTA-like” refers to substituted derivatives of BAPTA and BAPTA-analogues which retain the essential calcium-chelating characteristics of the parent (BAPTA) compound (see U.S. Pat. No. 4,603,209, issued to Tsien, R., et al., the entire contents of which patent are incorporated herein by reference). By this definition, “BAPTA-like” compounds include compounds such as quin-1, quin-2, stil-1, stil-2, indo-1, fura-1, fura-2, fura-3, and derivatives thereof.

Quin-1 refers to 2-[[2-bis(carboxymethyl)amino]-5-methylphenoxy]methyl]-8-bis(carboxymethyl)amino]-quinoline. Quin-2 refers to 2-[[2-[bis(carboxymethyl)amino]-5-methylphenoxy]-6-methoxy-8-[bis(carboxymethyl)amino]quinoline. Stil-1 refers to 1-(2-amino-5-[2-(4-carboxyphenyl)-E-ethenyl-1]phenoxy)-2-(2′-amino-5′-methylphenoxy)ethane-N,N,N′,N′-tetraacetic acid. Stil-2 refers to 1-(2(2-amino-5-[(2-(4-N,N-dimethylaminosulfonylphenyl)-E-ethenyl-1-]phenoxy)2-(2′-amino-5′methylphenoxy)ethane-N,N,N′,N′-tetraacetic acid. Indo-1 refers to 1-(2-amino-5-[6-carboxyindolyl-2]1 -phenoxy)-2-(2′-amino-5′-methylphenoxy)ethane-N,N,N′,N′-tetraacetic acid. Fura-1 refers to 1-(2-(4-carboxyphenyl)-6-amino-benzofuran-5-oxy)-2-(2′amino-5′-methylphenoxy)ethane-N,N,N′,N′-tetraacetic acid. Fura-2 refers to 1-(2-(5′-carboxyoxazol-2′-yl)-6-aminobenzofuran-5-oxy)-2-(2′-amino-5′methylphenoxy)ethane-N,N,N′,N′-tetraacetic acid. Fura-3 refers to 1-(2-(4-cyanophenyl)-6-aminobenzofuran-5-oxy)-2-(2′amino-5′-methylphenoxy)ethane-N,N,N′,N′-tetraacetic acid. The chemical structures for the above-identified calcium chelators are illustrated in U.S. Pat. No. 4,603,209, the entire contents of which patent are incorporated herein by reference.

As used herein, the phrase “pharmaceutically acceptable esters” (of the intracellular chelators) refers to lipophillic, readily hydrolyzable esters which are used in the pharmaceutical industry, especially α-acyloxyalkyl esters. See generally, references Ferres, H., 1980 Chem. Ind. pp. 435-440, and Wermuth, C. G., 1980 Chem. Ind. pp. 433-435. In a preferred embodiment, the intracellular chelator is the acetoxymethyl ester of quin-2 (Tsien, R., et al. (1982) J. Cell. Biol. 94:325-334). Esterification transforms the hydrophilic chelator into a lipophillic derivative that passively crosses the plasma membrane, and once inside the cell, is cleaved to a cell-impermeant product by intracellular esterases. Additional examples of intracellular calcium chelators are described in “Handbook of fluorescent Probes and Research Chemicals,” 5th edition, distributed by Molecular Probes, Inc., Eugene, Oreg.

As used herein, the phrase “agents for inhibiting actin filament severing” also embraces agents which directly inhibit gelsolin severing by affecting the platelet polyphosphoinositides. Such agents include, for example, phosphotidylinositol 4-phosphate, phosphotidylinositol 4,5-bisphosphate and compounds structurally related thereto (Janmey, P. and Stossel, T., 1987 Nature 325:362-365; Janmey, P., et al., 1987 J. Biol. Chem. 262:12228-12232).

The second agent inhibits barbed end actin polymerization. As used herein, “actin polymerization” refers to the process by which actin monomers (“G-actin”) are assembled onto the fast-growing (“barbed end”) of actin filaments (“F-actin”). Exemplary inhibitors of actin polymerization include the class of fungal metabolites known as the cytochalasins and derivatives thereof (see e.g., “Biochemicals and Organic Compounds for Research and Diagnostic Reagents” 1992, Sigma Chemical Company, St. Louis, Mo.). Cytochalasin B is one of the best characterized of the cytochalasins. It is believed that the cytochalasins inhibit actin polymerization by competing with endogenous barbed end capping agents, e.g., gelsolin, and reducing the rate of monomer addition to the barbed end of growing filaments.

As used herein, the “agents for inhibiting actin polymerization” include inhibitors having a similar mode of inhibition as the cytochalasins (presumably ppI-induced actin assembly), as well as inhibitors of actin polymerization having alternative mechanisms. Other xenobiotics having similar actions as the cytochalasins on platelet actin assembly include the Coelenterate-derived alkaloids, the latrunculins; the mushroom toxins, the virotoxins; and chaetoglobosins from different fungal species. Additional agents known to inhibit actin polymerization include actin monomer-binding proteins, profilin, thymosin, the vitamin D-binding protein (Gc globulin), DNAase I, actin-sequestering protein-56 (ASP-56), and the domain 1 fragments of gelsolin and other actin filament-binding proteins (see e.g., references cited in U.S. Pat. Nos. 5,876,676; 5,576,213; and 5,358,844. In addition, ADP-ribosylated actin reportedly acts like a barbed end-capping protein and inhibits barbed end actin assembly (Aktories, K. and Wegner, A., 1989 J. Cell Biol. 109:1385). Accordingly, agents which ADP-ribosylate actin, e.g., certain bacterial toxins such as Clostridium botulinum C2 and iota toxins, are embraced within the meaning of agents for inhibiting actin polymerization. Regardless of the mechanism of inhibition, the actin polymerization inhibitors have in common the ability to penetrate the plasma membrane.

In a preferred embodiment, quin-2AM is the first agent for inhibiting actin filament severing and cytochalasin B or dihydro-cytochalasin B is the second agent for inhibiting actin polymerization.

The foregoing methods and compositions are illustrative of the processes and agents for preparing chilled platelets that can be treated in accordance with the compositions and methods of the instant invention to prepare platelets which exhibit an increased circulatory time in vivo. Alternatively, chilled platelets which have not been contacted with the above-described agents for inhibiting cold induced platelet activation can be used in accordance with the compositions and methods of the instant invention.

According to a first aspect of the invention, a first method for identifying a “platelet clearance antagonist” is provided. The method involves contacting a chilled platelet with a liver macrophage (e.g., Kuppfer cell) in the presence and in the absence of a test molecule (e.g., a molecular library); and detecting binding of the chilled platelet to the liver macrophage, wherein a reduction in the binding in the presence of the test molecule relative to the binding in the absence of the test molecule indicates that the test molecule is a platelet clearance antagonist.

Binding assays to detect the binding of one cell to another or of a cellular component to a cell or other cellular component include in vitro and in vivo assays, e.g., FACs analysis. See also the Examples for a phagocytosis assay which detects binding of platelets to macrophages by detecting phagocytosis of the platelets by the macrophages; and U.S. Pat. No. 5,610,281 for an exemplary binding assay between two cell types or isolated ligands/receptors thereof.

As used herein, a “platelet clearance antagonist” refers to an agent which: (1) binds to a platelet ligand or binds to a liver macrophage receptor; and (2) prevents binding of the platelet ligand to the liver macrophage receptor. As used herein, platelet clearance antagonists which bind to platelet ligands are referred to as “platelet antagonists”; platelet clearance antagonists which bind to liver macrophage receptors are referred to as “receptor antagonists”. Exemplary platelet antagonists and receptor antagonists bind to the platelet ligands and liver macrophage receptors, respectively, provided in Table 1. Although not wishing to be bound to any particular theory or mechanism, it is believed that the binding of the platelet ligand to the liver macrophage receptor is involved in the differential clearance of chilled platelets compared to senescent platelets. Accordingly, platelet clearance antagonists are useful for prolonging the survival of chilled platelets in vivo. A particularly preferred class of platelet clearance antagonists are antibodies or fragments thereof which selectively bind to platelet ligands or liver macrophage receptors and, thereby, inhibit the binding of these molecules to their respective counter receptors.

Additional methods for identifying platelet clearance antagonists are provided as discussed below. In these and other aspects, it is to be understood that these definitions apply to the other aspects of the invention as disclosed herein.

In certain embodiments, the platelet antagonist selectively binds to a platelet ligand identified in Table 1. Preferably, the platelet antagonist selectively binds to the platelet ligand that is vWfR or a subunit thereof (GPIb α, GPIb β, GPIX and GPV). Most preferably, the platelet antagonist selectively binds to GPIb α. The platelet antagonist can be any type of binding molecule, e.g., an antibody or fragment thereof, provided that the platelet antagonist selectively binds to the platelet ligand and inhibits binding of a chilled platelet to a liver macrophage (e.g., Kuppfer cell).

According to still another aspect of the invention, a second method for identifying a platelet clearance antagonist is provided. The method involves contacting an isolated platelet ligand with a liver macrophage (e.g., Kuppfer cell) in the presence and in the absence of a test molecule (e.g., library molecule(s), antibodies, etc.); and detecting binding of the platelet ligand to the liver macrophage, wherein a reduction in the binding in the presence of the test molecule relative to the binding in the absence of the test molecule indicates that the test molecule is a platelet clearance antagonist.

According to yet another aspect of the invention, a third method for identifying a platelet clearance antagonist is provided. The method involves contacting an isolated platelet ligand with an isolated liver macrophage (e.g., Kuppfer cell) receptor in the presence and in the absence of a test molecule; and detecting binding of the platelet ligand with the liver macrophage receptor, wherein a reduction in the binding in the presence of the test molecule relative to the binding in the absence of the test molecule indicates that the test molecule is a platelet clearance antagonist.

According to a still further aspect of the invention, a fourth method for identifying a platelet clearance antagonist is provided. The method involves contacting a chilled platelet with an isolated liver macrophage (e.g., Kuppfer cell) receptor in the presence and in the absence of a test molecule; and detecting binding of the chilled platelet with the liver cell receptor, wherein a reduction in the binding in the presence of the test molecule relative to the binding in the absence of the test molecule indicates that the test molecule is a platelet clearance antagonist.

In certain of the embodiments of the screening methods and other methods disclosed below, the platelet clearance antagonist is a platelet antagonist (e.g., the platelet antagonist binds to a platelet ligand selected from the group of platelet ligands provided in Table 1). As noted above, a preferred platelet antagonist binds to a platelet ligand that is vWfR or a subunit thereof. In these and/or other embodiments, the platelet clearance antagonist is a liver macrophage receptor antagonist (e.g., the liver macrophage receptor antagonist binds to a liver macrophage receptor such as the group of liver macrophage receptors provided in Table 1.) Preferably, the liver macrophage receptor is expressed by Kuppfer cells. As noted above, a preferred liver macrophage receptor antagonist of the invention binds to αMβ2.

The screening methods of the invention are useful for identifying test molecules which are platelet clearance antagonists. Such test molecules can be rationally designed or identified in mixtures of molecules, such as combinatorial libraries.

According to another aspect of the invention, a method for preparing platelets for transfusion is provided. The method involves contacting a chilled platelet with a platelet antagonist under conditions to permit the chilled platelet antagonist to bind to a ligand on the chilled platelet and, thereby, form a platelet antagonist-treated platelet. The contacting can be performed before, during or after chilling of the platelets; although it is preferable to contact the platelets with the platelet antagonist after the platelets are chilled. Optionally, contacting can be performed while the platelets are contained in a platelet bag. Preferably, the platelet antagonist selectively binds to a platelet ligand identified in Table 1. More preferably, the platelet antagonist selectively binds to vWfR or a subunit thereof. In these and other embodiments, the chilled platelets, optionally, are treated with one or both of a first agent for inhibiting actin filament severing (e.g., an intracellular calcium chelator such as Quin-1) and a second agent for inhibiting actin polymerization (e.g., a cytochalasin).

In certain embodiments, the method of preparing the platelets for transfusion further includes the step of separating the platelet antagonist-treated platelet from the platelet antagonist that has not bound to the chilled platelet. Such methods can easily be performed in accordance with standard procedures for separating cells from non-cell components (e.g., size exclusion based methods, including centrifugation).

Antibodies and binding fragments thereof are a preferred class of platelet clearance antagonists. Antibodies include polyclonal and monoclonal antibodies, prepared according to conventional methodology. Exemplary antibodies and fragments thereof are derived from the antibodies illustrated in FIG. 14.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The invention involves binding polypeptides of numerous size and type that bind selectively to the platelet ligands or liver macrophage receptors and, thereby, inhibit binding of platelets to macrophages. These binding polypeptides also may be derived from sources other than antibody technology. For example, such polypeptide binding agents can be provided by degenerate peptide libraries which can be readily prepared in solution, in immobilized form, as bacterial flagella peptide display libraries or as phage display libraries. Combinatorial libraries also can be synthesized of peptides containing one or more amino acids. Libraries further can be synthesized of peptides and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which inhibit the binding of the platelet ligands to the liver macrophage receptors. This selection process can be accomplished in a one step method (i.e., by screening the library directly for molecules which inhibit this binding) or in a multi-step process (e.g., by screening the library for molecules which bind to the platelet ligand and/or the macrophage receptor and, thereafter, testing such binding molecules to determine whether they inhibit platelet binding to macrophage). This process can be repeated through several cycles of reselection of phage that inhibit binding of the platelet ligand to the liver macrophage receptors (or binding to these components, followed by at least one screening step to identify library molecules that inhibit platelet binding to macrophage). Repeated rounds lead to enrichment of phage bearing particular sequences. DNA sequence analysis can be conducted to identify the sequences of the expressed polypeptides. The minimal linear portion of the sequence that binds to the platelet ligands or liver macrophage receptors can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the platelet ligands or liver macrophage receptors. Thus, the platelet ligands or liver macrophage receptors of the invention, or a fragment thereof, or complexes of platelet ligands or liver macrophage receptors can be used to screen peptide libraries, including phage display libraries, to identify and select peptide binding polypeptides that selectively bind to the platelet ligands or liver macrophage receptors of the invention. Such molecules can be used, as described, for screening assays, for purification protocols, for interfering directly with the functioning of platelet ligands or liver macrophage receptors and for other purposes that will be apparent to those of ordinary skill in the art.

Platelet ligands or liver macrophage receptors, or fragments thereof, also can be used to isolate naturally occurring, polypeptide binding partners which may associate with the platelet ligands or liver macrophage receptors in the membrane of a platelet or cell. Isolation of binding partners may be performed according to well-known methods. For example, isolated platelet ligands or liver macrophage receptors can be attached to a substrate, and then a solution suspected of containing platelet ligands or liver macrophage receptors binding partner may be applied to the substrate. If the binding partner for platelet ligands or liver macrophage receptors is present in the solution, then it will bind to the substrate-bound platelet ligands or liver macrophage receptors, respectively. The binding partner then may be isolated. Other proteins which are binding partners for platelet ligands or liver macrophage receptors, may be isolated by similar methods without undue experimentation.

The chilled platelets or the platelet antagonist-treated platelets are contacted with a liver macrophage receptor antagonist (e.g., to combine delivery of the platelets (untreated or treated with platelet antagonist and the receptor antagonist) prior to infusion. As noted above, a preferred liver macrophage receptor antagonist binds to a liver macrophage receptor selected from the group of liver macrophage receptors provided in Table 1. More preferably, the liver macrophage receptor antagonist binds to a liver macrophage receptor that is αMβ2.

The foregoing methods of preparation of platelets can include the further step of administering the platelet antagonist-treated platelet to a subject.

The invention also provides a method for forming a medicament. The method involves placing a plurality of one or more platelet clearance antagonists and, optionally, chilled platelets, in a pharmaceutically acceptable carrier. In certain embodiments, the platelet clearance antagonist is a platelet antagonist. In these and other embodiments, the platelet clearance antagonist is a liver macrophage receptor antagonist.

According to another aspect of the invention, a composition containing a plurality of platelets; and one or more platelet clearance antagonists as described herein, is provided. The platelet clearance antagonists can include a platelet antagonist and/or a liver macrophage receptor antagonist. The preferred platelet antagonists selectively bind to vWfR or a subunit thereof. The preferred liver macrophage receptor antagonists selectively bind to αMβ2. Optionally, the composition further includes a pharmaceutically acceptable carrier. In these and other embodiments, the composition optionally is contained in a platelet bag.

In a preferred embodiment, the platelets are collected into a platelet pack or bag according to standard methods known to one of skill in the art. Typically, blood from a donor is drawn into a primary bag which may be joined to at least one satellite bag, all of which bags are connected and sterilized before use. In a preferred embodiment, the platelets are concentrated (e.g. by centrifugation) and the plasma and red blood cells are drawn off into separate satellite bags (to avoid modification of these clinically valuable fractions) prior to sequentially adding one or more platelet clearance antagonists. Platelet concentration prior to treatment also minimizes the amounts of the platelet clearance antagonists required, thereby minimizing the maximum amounts of this agent that may be eventually infused into the patient.

In a most preferred embodiment, the platelet clearance antagonist(s) is contacted with the platelets in a closed system, e.g. a sterile, sealed platelet pack, so as to avoid microbial contamination. Typically, a venipuncture conduit is the only opening in the pack during platelet procurement or transfusion. Accordingly, to maintain a closed system during treatment of the platelets with the platelet clearance antagonists, the antagonist(s) are placed in a relatively small, sterile container which is attached to the platelet pack by a sterile connection tube (see e.g., U.S. Pat. No. 4,412,835, the contents of which are incorporated herein by reference). The connection tube is reversibly sealed according to methods known to those of skill in the art. After the platelets are concentrated, e.g. by allowing the platelets to settle and squeezing the plasma out of the primary pack and into a satellite bag according to standard practice, the seal to the container(s) including the platelet clearance antagonist(s) is opened and the antagonist(s) are introduced into the platelet pack. In a preferred embodiment, the platelet antagonist and the liver macrophage receptor antagonists are contained in separate containers having separate resealable connection tubes to permit the sequential addition of these platelet clearance antagonists to the platelet concentrate, as needed.

The platelet antagonist-treated platelets are stored at a reduced temperature that is less than standard platelet storage temperatures, e.g., less than about 22 degree C. In a preferred embodiment, the reduced temperature ranges from about 0 degree C. to about 4 degree C. In contrast to platelets stored at, for example, 22 degree C., platelets stored at reduced temperatures have substantially reduced metabolic activity. Thus, platelets stored at 4 degree C. are metabolically less active and therefore do not generate large amounts of CO₂ compared with platelets stored at, for example, 22 degree C. Dissolution of CO₂ in the platelet matrix reportedly results in a reduction in pH and a concomitant reduction in platelet viability. Accordingly, conventional platelet packs are formed of materials that are designed and constructed of a sufficiently permeable material to maximize gas transport into and out of the pack (O₂ in and CO₂ out). The prior art limitations in platelet pack design and construction are obviated by the instant invention, which permits storage of platelets at reduced temperatures, thereby substantially reducing platelet metabolism and diminishing the amount of CO₂ generated by the platelets during storage.

The platelet-containing compositions and/or platelet clearance antagonists of the invention optionally further include a pharmaceutically acceptable carrier. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

In certain embodiments, the composition includes both a first platelet clearance antagonist that is a platelet antagonist and a second platelet clearance antagonist that is a liver macrophage. Alternatively, the composition may contain one type of platelet clearance antagonist (e.g., a platelet antagonist or a liver macrophage receptor antagonist). The preferred platelet clearance antagonists and liver macrophage receptor antagonists are those which selectively binds to the ligands and receptors, respectively, identified in Table 1. More preferably, the platelet antagonist selectively binds to vWfR or a subunit thereof and the liver macrophage receptor antagonist selectively binds to αMβ2.

According to one aspect of the invention, a method for increasing platelet circulatory time is provided. The method involves administering to a subject in need of such treatment, one or more platelet clearance antagonists in an amount effective to increase platelet circulatory time in the subject. Optionally, the composition further includes a plurality of chilled platelets. In these and other embodiments, the platelets can be contained in a platelet bag to facilitate administration to the subject. Additionally or alternatively, platelet antagonist-treated platelets (i.e., platelets which have been treated with one or more platelet antagonists) can be administered to a subject. Optionally, unbound platelet antagonists are removed from the composition prior to administration of the platelet antagonist-treated platelets. Chilled platelets may be contained in the composition or separately administered to the subject.

According to yet another aspect of the invention, a method for treating a subject in need of platelets is provided. The method involves administering to the subject, a composition comprising: (1) a first composition containing: (a) a plurality of chilled platelets; and one or more platelet clearance antagonists; (2) a second composition containing: a plurality of platelet-antagonist-treated platelets; or (3) a third composition containing: a plurality of platelet lesion cleavage agent-treated (described below) platelets, wherein the first composition or the second composition or the third composition are administered in an amount effective to treat the subject. The preferred platelet clearance antagonists are as described above. Optionally, unbound platelet antagonists or cleavage agents are removed from the second composition prior to administration of the platelet antagonist-treated platelets. In these and other embodiments, the composition optionally further includes one or more liver macrophage receptor antagonists. Chilled platelets may be contained in the composition or separately administered to the subject. In these and other embodiments, the platelets can be contained in a platelet bag to facilitate administration to the subject.

According to yet another embodiment, a method for identifying a platelet lesion cleavage agent is provided. The method involves contacting a chilled platelet with a liver macrophage or receptor thereof (or isolated ligands and receptors, respectively, or fragments thereof) in the presence and in the absence of a test cleavage agent; and detecting binding of the chilled platelet to the liver macrophage, wherein a decrease in the binding in the presence of the test cleavage agent relative to the binding in the absence of the test cleavage agent indicates that the test molecule is a platelet lesion cleavage agent. Preferably, the test cleavage agent is selected from the group consisting of enzymes that cleave carbohydrates (e.g., a galactosidase, a glucosidase, a mannosidase) or that cleave enzymes. See, e.g., U.S. Pat. Nos. 4,330,619; 4,427,777; and 4,609,227 (the entire contents of which are incorporated herein by reference) for a description of the methods for removing type A and type B antigens from erythrocytes which employ antigen cleavage with a galactosidase and U.S. 5,671,135 for a description of an automated process that is useful for performing this and other cell surface molecule cleavage methods.

The conditions for contacting a chilled platelet of the invention with the particular cleavage enzyme are selected for the particular cleavage enzyme that is being used for the process. Such cleavage enzymes and conditions are known to those of ordinary skill in the art. Following enzyme treatment, the enzyme is removed from the platelets and the platelets are reequilibrated in a buffer which may also serve as a pharmaceutically acceptable carrier. Thereafter, the platelets can be used for transfusion therapy in accordance with standard procedures.

Binding of the chilled platelet to the liver macrophage can be detected in accordance with any of the above-described methods; however, a preferred method for detecting binding involves detecting phagocytosis of the chilled platelet by the liver macrophage. Although not wishing to be bound to any particular theory or mechanism, it is believed that chilling of platelets induces changes in the surface expression of platelet proteins such as vWfR or a subunit thereof, which play a role liver macrophage clearance. Accordingly, it is believed that the cleavage of such aberrant surface proteins removes or modifies chilled platelet ligands that are essential for liver macrophage receptor recognition. Thus, a method which further involves contacting the platelet lesion cleavage agent with a chilled platelet permits the platelet lesion cleavage agent to cleave a platelet lesion on a chilled platelet and, thereby, forming platelets which have prolonged survival in vivo. The invention further embraces platelets that are prepared in accordance with this method.

The following Examples are illustrative only and are not intended to limit the scope of the invention in any way.

### EXAMPLES

Introduction to the Examples:

We have used a murine system for measuring platelet survival. More specifically, we have transfused mouse or human platelets that are fluorescently or radioactively labeled into the mice and measured their survival and their sites of deposition. In this system unchilled platelets circulated for several days and accumulated in the spleen. In contrast, chilled platelets were rapidly cleared, even if maintained in a discoid shape by treatment with agents which inhibit cold-induced platelet activation, and were deposited in the liver. A high-resolution imaging assay and a FACS analysis of cells removed from the livers showed that these platelets associated with liver macrophages (Kuppfer cells). Although not wishing to be bound to any particular theory or mechanism, we believe that cold alters the major platelet glycoprotein GPIb so that it interacts with the major macrophage β2 integrin (αMβ2), thereby facilitating chilled platelet clearance by liver macrophages.

The experiments described below include methods for further characterizing the molecular interactions underlying chilled platelet clearance. Thus, for example, monoclonal antibodies to representative platelet ligands and liver macrophage receptors and/or knockout mice missing the respective receptors, can be used to further characterize the essential molecular interactions involved in liver mediated chilled platelet clearance. In view of our discoveries regarding chilled platelet clearance, the invention further provides screening assays to identify platelet clearance antagonists which maximize inhibition of clearance while minimally inhibiting platelet function.

### Example 1

Mechanisms of Platelet Clearance.

The following experiments provide evidence that platelet clearance after chilling <14 degree C. induces a change in the platelet surface that leads to recognition and ingestion by macrophages. The primary site where this occurs is the liver. Based on these observations, the following additional experiments have been designed and performed to further characterize key determinants in platelet clearance by:

1. Confirming that the liver is the site where platelets are removed in mammals after chilling and that the liver macrophages, or Kupffer cells, are engorged with the platelets. The extent to which the methods reported to protect against loss of platelet function with cooling modify the kinetics or location of platelet removal also is determined.

2. Establishing an in vitro macrophage-based phagocytic assay that is selective for chilled platelets (described in detail below).

3. Identifying the cellular mechanism(s) of phagocytosis of chilled platelets, particularly macrophage receptor(s) such as amp2 that are engaged by chilled platelets and mediate the ingestion of platelets. After identification in the phagocytic assays, circulation studies are used in mice to confirm that blockage of or lack of (knockout animals) this receptor prohibits the clearance of cold-treated platelets;

4. Identifying the change that occurs in the platelet surface that leads to recognition by macrophages.

It has been recently recognized that activation per se does not necessarily diminish platelet survival. Platelets transfused in both monkeys and mice after activation in vitro by thrombin have circulation times similar to normal platelets (Michelson, A. et al., Proc. Natl. Acad. Sci., U.S.A. 93:11877-11882; Ware, J. et al., Proc. Natl. Acad. Sci., USA 97:2803-2808). These surprising results, in combination with our studies below, clearly indicate that cold induced clearance is mediated by mechanisms other than shape change and P-selectin upregulation. Chilling of platelets isolated from P-selectin knockout mice still elicits rapid clearance, apparently eliminating a role for P-selectin in this process. This result is surprising since P-selectin is intimately involved in the primary adherence reaction of platelets to blood leukocytes. The mechanisms of platelet birth and death have not been resolved. One-seventh of the total human platelet mass is removed and replaced daily. The mechanisms of platelet clearance are the focus of the experiments described herein.

Lessons Learned from Studies on RBC Clearance.

It is generally believed that aged or damaged RBCs are recognized and removed from blood by scavenger pathways that include alterations in surface carbohydrates (Vaysse, J. et al., Proc. Natl. Acad. Sci., USA 83:1339), adherence of anti-receptor antibodies (Lutz, H. et al., Blood Cells 14:175), or loss of membrane lipid asymmetry (Connor, J. et al., J. Biol. Chem. 269:2399; McEvoy, L. et al., Proc. Natl. Acad. Sci., USA 83:3311). These cellular alterations lead to recognition by macrophages in the spleen and liver, phagocytosis and removal. Macrophage receptors implicated in this process include the scavenger receptors (Terpstra, V. et al., Blood 95:2157-2163), mannose (Horn, s. et al., Biochem. Pharmacol. 39:775), Fc, and integrins, e.g. complement receptors.

Dissecting the signals that lead to platelet clearance must be regarded as a more formidable task to those defined for the senescent erythrocyte. Platelets undergo a variety of physiological changes not seen in RBCs. Platelets can change shape, secrete, insert new surface receptors, regulate the activities of exposed surface receptors, move receptors into the open canalicular system, and expose phosphatidylserine. Quite simply, the mechanism of normal clearance of platelets is unknown, and whether platelet removal induced by cooling occurs by the same or different mechanisms has not been investigated. Of these two processes (normal versus cold), cold may in fact be the easiest to address empirically, as many of the changes that occur with platelet aging do not occur in the brief 1 hr cooling time that is required to elicit rapid clearance. For example, cooling for short times does not induce phosphatidylserine transfer to the cell surface, microvesicularization, or secretion.

Platelet Surface Receptors and Clearance.

Resting platelets are uniquely constructed subcellular particles having a reproducible complement of receptors that densely coat the plasma membrane. The major molecules expressed on the cell surface are MA (αIIBβ3, GPIIb/IIIa), von Willibrand factors (GPIbαβ/IX/V) (vWfR), and GPIV (CD36-thrombospondin and FA scavenger receptor) of which there are ˜50,000, 25,000, and 10,000 molecules, respectively. Many other molecules are also expressed on the surface but are present at <1000 copies per platelet including signaling receptors and glycoproteins that may be involved in certain aspects of the cell clearance mechanisms. Although storage at room temperature in blood bags has been shown to lead to proteolysis of many of the major platelet glycoproteins, cold storage prohibits these proteolytic events. Hence, cold-induced clearance, if mediated at the cell surface, could be caused by either: (a) the removal of a protective agent, as has been recently proposed for CD47 (Oldenborg, P. -A. et al., Science 288:2051-2054): (b) the appearance of new molecules on the platelet surface that signal for cell removal; or (c) by certain changes in the state of molecules present on platelet surface to positively influence clearance.

One interesting platelet receptor-macrophage co-receptor pair recently identified that we believe is involved in clearance is vWfR and, αMβ2 (alternatively referred to in the literature as Mac-1 or CD11b/CD18) (Lopez, J. et al., Blood, Simon, d. et al., J. Exp. Med. 192). MAC1 binds to the GPIbα chain of the vWfR receptor. This interaction will immobilize leukocytes on GP1bα-coated surfaces and does not occur in leukocytes from mice lacking Mac-1 or after treatment of platelets with mocarhagin, a snake venom metalloprotease that specifically removes the N-T of GP1bα.

Down-regulation of vWfR following platelet activation requires actin remodeling. In resting platelets, the receptor complex for the vWf in the plasma membrane is linked to underlying actin filaments by filamin (actin-binding protein-280) molecules in an interaction that occurs between the cytoplasmic tail of GP1α (Andrews, R. et al., J. Biol. Chem. 266:7144-7147; Andrews, R. et al., J. Cell Biol. 267:18605-18611) and the carboxyl terminus of filamin (Aakhus, Al. et al., Throm. Haem. 67:252-257; Ezzell, R. et al., J. Biol. Chem. 263:13303-13309). The vWF receptor is a complex of 4 polypeptides: GPIbα, GPIbβ, GPIX and GPV (Li, C. J. Dong et al., J. Biol. Chem. 270:16302-16307; Lopez, J. et al., Proc. Natl. Acad. Sci., USA 85:2135-2139; Lopez, J. et al., J. Biol. Chem. 269:23716-23721), present at ˜25000-30000 copies per platelet. Because it is connected to underlying actin filaments, vWiR is not randomly dispersed over the platelet surface, but instead is aligned into linear arrays (Kovacsovics, T. et al, Blood 87:618-629).

Platelet activation causes a dynamic redistribution of vWfR complexes and of several other cell surface receptors. Shortly after stimulation, the surface expression of P-selectin (Stenberg, P., et al., J. Cell Biol. 101:880) and α_{IIb}β₃ (Wencel-Drake, J. D. et al., Am. J. Pathol. 124:324-334) increase (upregulation) as a result of a-granule fusion with the plasma membrane. Not only does the surface content of α_{IIb}β₃ increase during activation, but also these receptors, functionally cryptic for ligand binding in the resting cell, convert into active forms that can bind fibrinogen and other targets. In marked contrast to the increased content of these two receptors on the active cell surface, there is a progressive loss of vWfR from the cell surface (George, J. et al., Blood 71:1253-1259; Hourdille, P. et al., Blood 76:1503-1513; Lu, H. et al., Br. J. Haemat 85:116-123; Michelson, A. et al., J. Clin. Invest. 81:1734-1740; Michelson, A. D. et al., Blood 83:3562-3573; Michelson, A. D. et al., Blood 77:770-779). Removal of vWfR from the surface occurs through a centrifugal aggregation of it into the cell center, a process similar in many aspects to the capping of crosslinked receptors observed in other cells, although markedly different because crosslinking of vWfR is unnecessary. In a cell suspension, vWfR aggregates into the center of the cell and becomes sequestered in the OCS (Cramer, E. M. et al., Blood 77:694-699; Hourdill, P. et al., Blood 79:2011-2021; Hourdille, P. et al., Blood 76:1503-1513) and hence becomes inaccessible to antibodies (George, J. et al., Blood 71:1253-1259; Hourdille, P. et al., Blood 76:1503-1513; Lu, H. et al., Br. J. Haemat 85:116-123; Michelson, A. et al., J. Clin. Invest. 81:1734-1740; Michelson, A. D. et al., Blood 83:3562-3573; Michelson, A. D. et al., Blood 77:770-779).

The molecular mechanism of vWfR redistribution has not been completely established, but it is known that rearrangements of the actin cytoskeleton, actin assembly, and myosin II activation are necessary. Centralization of vWfR is inhibited by cytochalasin, which prevents actin filament assembly, and by loading the cells with calcium chelators (Kovacsovics, T. et al, Blood 87:618-629). Chelation prevents a calcium-dependent, gelsolin-mediated filament fragmentation that normally precedes actin assembly and which allows the membrane skeleton to remodel. The physiological relevance of vWfR aggregation has not been established nor is the response of the receptor well defined in the cold. In general, experiments reported thus far indicate that vWfR remains on the surface of chilled platelets, although some investigators have reported vWfR to be slowly lost in the cold as microvesicles are shed (Bode, A. et al., Transfusion 34:690-696). Our studies show only small decreases (˜4%) in vWfR on the surface of the chilled platelet and for these to occur, a minimum of 1 day of incubation in the cold is required. This decrease is somewhat less when shape change is prevented in chilled platelets by the addition of cytochalasin B (CB) and EGTA-AM loading (Winokur, R. et al., Blood 85:1796-1804). We believe that the presence of this receptor on the surface in an altered form may be one of the reasons why chilled platelets are cleared while activated platelets remain in the circulation.

Formulation for Preservation of Platelet Shape in the Cold and Testing Survivability.

Based on our studies of platelet activation by thrombin and other stimuli (described in more detail below) we had selected a mixture of cytochalasin B (CB) and the intracellular calcium chelator Quin 2 to prevent platelet shape change during chilling (Winokur, R. et al., Blood 85:1796-1804) and U.S. Pat. Nos. 5,358,844 and 5,876,676). We had shown that this combination prevented the disc to spiny sphere conversion of platelets chilled to 4° C. and subsequently re-warmed to 37° C. In addition, we observed that human platelets so treated and then chilled and re-warmed circulated freely and did not roll on venules visualized in mouse mesenteric lymph nodes, whereas untreated chilled and re-warmed platelets rolled. The platelets were functional, because infusion of TRAP (which does not activate mouse thrombin receptors) into the mice caused chilled preserved circulating platelets to begin rolling.

We found that in the presence of cytochalasin B and EGTA-AM, both added to gel-filtered human platelets at 2M, sustained nearly full thrombin-induced platelet aggregation activity for up to three weeks of storage at 4° C. followed by re-warming.

We next examined the effect of cold preservation in platelet-rich plasma (PRP). In contrast to the nice uniformly discoid platelets achieved by gel filtration, platelets in PRP appeared very heterogeneous in shape. Since the classical literature on platelet preservation emphasized how discoid shape was the best indication of in vivo survivability of platelets kept at room temperature, we were surprised by this observation. We concluded that the optics of light microscopy available at the time that those descriptions were made (the 1960s and 1970s) might have been more forgiving than what we use today. We eventually were able to convince blinded observers that the somewhat rattier platelets in PRP do clearly change shape in the cold and aggregate, and that the preservation technique largely prevents those effects. We then proceeded to optimize the process of preservation (a sequential addition of EGTA-AM (followed by CB) and showed that we could preserve discoid shapes of platelets in miniaturized blood storage bags.

Mechanism of Actin Assembly.

We studied the biochemical and structural basis for the cold-induced shape transformation in human platelets and found it to require net actin assembly. To understand the signals involved in this process, we applied a permeabilization scheme to the cooling process that we have used to dissect PAR-1 mediated actin assembly. In this approach, platelets are first permeabilized with 0.25% octylglucoside (OG), then chilled to 4° C. for 5 min and rewarmed to 37° C. Resting platelets remained discoid at 37° C. after permeabilization with OG but changed shape when chilled. Chilled OG-permeant platelets have blebs on their surfaces and elaborate filopodial processes. Rewarming (RW) does not alleviate these shape changes. Rewarming is required to assay filament end numbers, because we discovered that OG-treated platelets reseal in the cold, but become permeable again when rewarmed. We assayed the number of actin filament barbed ends biochemically by monitoring the acceleration of pyrene-actin polymerization rates fluorometrically in the presence and absence of CB (Hartwig, J. et al., Cell 82:643-653). Extraction of OG-treated platelets with Triton X-100 (TX) revealed that cold alone induces barbed end formation; chilling of permeant platelets leads to the production/exposure of 200 barbed ends/cell demonstrating that these permeant cells retain their response to cold. This experimental system demonstrates that cooling leads to barbed end exposure and allows us to probe the process through the addition of inhibitory reagents not normally able to penetrate into the cells.

The Signal for Barbed end Exposure in Platelets.

Platelets activated through the PAR-1 receptor protrude both filopodia and lamellipods when attached to surfaces. Filopodial assembly generally occurs first and is temporally followed by cell spreading although cells can spread without first making filopods. Actin assembles into filaments near the plasma membrane to drive these protrusions outward. Hence, the signals that cause actin assembly, and shape change, locate at the cytoplasmic face of the plasma membrane. These signals lead to exposure of barbed ends to initiate actin filament assembly by both releasing gelsolin and other capping proteins from the barbed ends of actin and by activating the Arp2/3 complex to nucleate filament assembly de novo from monomers. Work thus far from our laboratory and others has shown that lipids of the phosphoinositide (ppl) family inactivate proteins that cap the barbed ends of actin filaments (Hartwig, J. et al., J. Biol. Chem. 271:32986-32993) and activate the WASP family of Arp2/3 regulatory proteins. Phosphoinositides phosphorylated in the 3, 4, and 5 positions on their inositol ring can bind and inactivate gelsolin (Hartwig, J. et al., J. Biol. Chem. 271:32986-32993). Resting platelets have ˜200 μM each of phosphatidylinositol 4-monophosphate (PI₄P) and phosphatidylinositol 4,5-bisphosphate (PI₄₅P₂) in their membranes, equivalent to ˜2% of the total membrane lipid. Since these ppI concentrations are more than sufficient to inactivate gelsolin and other capping proteins (where 10-50 μM ppl is maximally effective), the bulk of the inositol head groups at the cytoplasmic membrane surface of the resting cell must be inaccessible to barbed end capping proteins, presumably sequestered by other inositol lipid binding proteins. After ligation of the thrombin receptor PAR-1, the most potent of the platelet serpentine receptors, the mass concentrations of ppls in the membrane change rapidly. A rapid activation of phospholipase C_{β} leads to an initial hydrolysis of PI_{4,5}P₂ to diacylglycerol (DAG) and inositol trisphosphate (IP₃). Although the net mass of PI₄P and PI_{4,5}P₂ decreases initially, the activity of PI-4 and PI-5 kinases are robustly simulated, and D4 and D5-containing ppI mass in the plasma membrane is restored and, in fact, increases by 20-40% over the resting level 30 sec following receptor ligation (Hartwig, J. et al., Cell 82:643-653; Hartwig, J. et al., J. Biol. Chem. 271:32986-32993). PI-3 kinase is also rapidly activated. Since the D3-containing ppis are not substrates for phospholipase C family, their mass in the membrane increases 10-25 fold. Although dramatically increased relative to rest, the D3-containing ppI still represent only a small fraction of the total membrane ppl mass. D3 containing ppI are essential downstream messengers for certain platelet receptors to signal to actin, in particular the FcγRIIA and αIIB/β3 receptors, but not for actin assembly and shape change mediated by the PAR-1 receptor.

While the pathway to ppI degradation from the PAR-I receptor is well understood, signals leading to ppl synthesis are just being defined. Calcium release is mediated by soluble IP₃ released when phospholipase C_{β} is activated by the By-trimeric G-protein subunit. PpI synthesis, on the other hand, requires the activation of the rho family GTPases rac1 and rac2 in platelets: the conversion of racs to their GTP-forms occurs with kinetics that mirrors phospholipid synthesis (Azim, A. et al., Blood 95:959-964). GTP-rac moves to the plasma membrane where it binds to PI-5 Iα and PI-3 kinases (Tolias, K. et al., Curr. Biol. 10:153-156). PI-5 Iα kinase binds both GDP-rac and GTP-rac although its activity in cells is stimulated by only GTP-rac. PI-3 kinase binds and is activated by only the GTP-form. As mentioned above, the activity of PI-3 kinases is not required for actin assembly initiated through the PAR-1 receptor (Tolias, K., et al., J. Biol Chem. 270:17656-17659).

Actin Assembly Induced by Chilling.

Based on the results summarized in the previous paragraph, we hypothesized that chilling might cause a phase transition in the plasma membrane to aggregate the ppis, PI₄P and PI₄₅P₂, in the membrane of the platelet. Clustering can experimentally potentate the activity/exposure of the phospholipids in the cytoplasmic membrane surface. PI_{4,5}P₂ would dissociate proteins capping the barbed ends of filaments of the cell and/or activate the Arp2/3 actin nucleating complex. We first investigated the role of PI_{4,5}P₂ in this process by treating the OG-permeant platelets with a peptide that binds and sequesters ppIs before chilling. 20-30 μM peptide inhibited barbed end exposure when OG-permeant platelets are cooled to 4° C. These data show that ppls are clearly involved in the actin assembly reaction that distorts the shape of the platelet in the cold. Others have provided evidence for a membrane phase change in chilled platelets (Tablin, F., et al., J. Cell. Phys. 168:305-313).

When platelets are activated by agonists at 37° C., ppl production is downstream of receptors and small GTPases and can be inhibited by the small GTPase antagonists GDPβS or negative dominant GTPases. Experiments in chilled OG-permeabilized platelets, however, reveal actin assembly to be uncoupled from GTP and GTPases. Our result showed that the non-hydrolizable GTPγS and GDPβS analogs have no effect on the number of barbed ends exposed upon cooling. In agreement with this finding, NI1rac or N17cdc42 (negative dominant forms) also failed to inhibit barbed end exposure when the OG-permeant platelets were cooled. As a control, GDPβS was used as a potent inhibitor of PAR-1-mediated signaling to actin, as we have previously reported (Hartwig, J. et al., Cell 82:643-653). Therefore, these data support our hypothesis that lipid rearrangements, uncoupled from receptors, are inducing actin assembly and causing a shape change in the cold.

The Targets of ppIs in Chilled Platelets.

Known effector proteins of ppls in activated cells include barbed capping proteins and the activation of the Arp2/3 complex by WASP-ppI complexes. We first determined the role of the Arp2/3 nucleation complex in cold-induced actin assembly in the OG-permeant cells. Experiments were based on constructs derived from the carboxyl-T of N-WASP which act as either dominant negative inhibitors of Arp2/3 (a construct called CA) or as constitutive activators (a construct called VCA). The addition of 3 μM CA (a maximal dose based on studies in PAR-1 activated platelets) to the permeant-platelets inhibited the cold response by ˜40%, whereas VCA potently activated barbed end exposure to a much larger extent than chilling alone. From these data, we conclude that Arp2/3 activation contributes half of the actin assembly in chilled platelets.

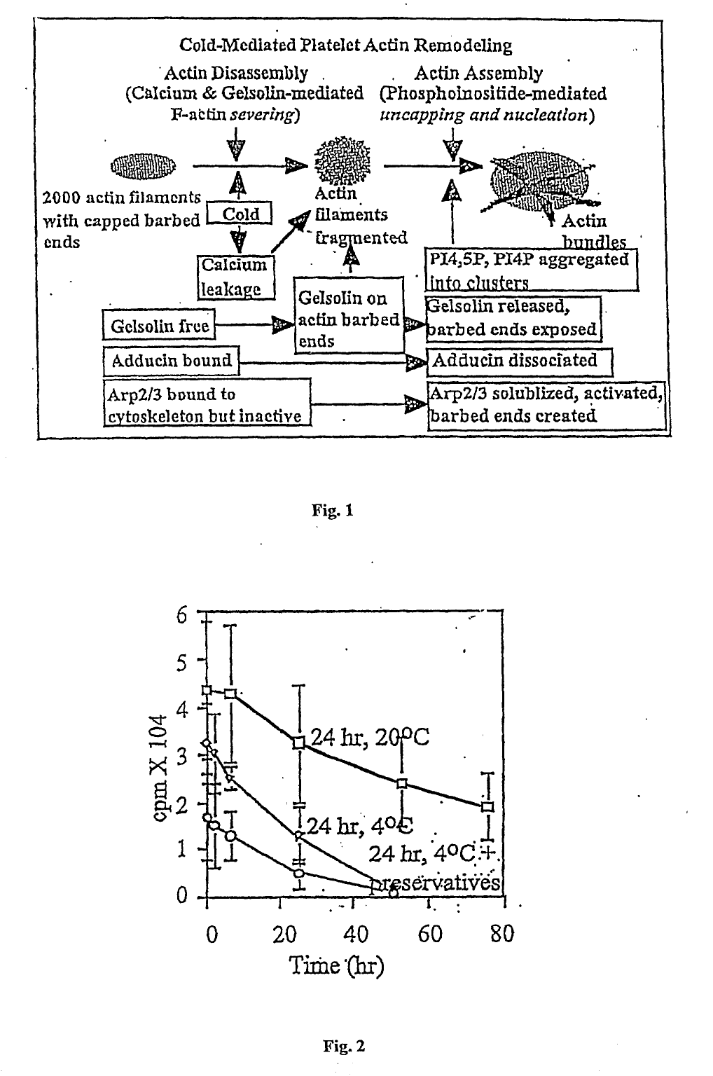

Other critical components of the cold response are the barbed end capping proteins, gelsolin and adducin, a conclusion based on three observations. First, gelsolin transiently associates then dissociates from the actin cytoskeleton of chilled platelets. In resting platelets, gelsolin is entirely soluble. Binding to actin is induced by calcium, and like-PAR-1 activation, gelsolin first associates then dissociates from actin in a similar fashion following chilling. 6. Second, gelsolin null platelets have a blunted shape change response to chilling compared to wild-type mouse platelets that contain gelsolin. Association of gelsolin with actin is temporally correlated with actin filament fragmentation, and platelets from gelsolin knockout mice do not display normal filament fragmentation. Third, cooling dissociates adducin from the actin cytoskeleton. Our studies on platelet adducin have revealed 70-80% of the total adducin is bound to the resting actin cytoskeleton where it caps the barbed ends of actin filaments. Dissociation of adducin would be expected to expose barbed ends and to contribute to the actin assembly reaction induced by chilling. In summary, all data that we have accumulated thus far lead us to construct the following diagram for cold-induced shape change (FIG. 1). The initial response to cold is that cytosolic calcium increases. This activates gelsolin to bind and sever actin filaments. Cold then induces ppI aggregation, triggering gelsolin and adducin dissociation from filament ends and perhaps WASP-activation. PpI-WASP would then bind to and activate the Arp2/3 complex providing a second source of nucleation sites to stimulate actin filament assembly in the cold.

These results justify the rationale for the use of cytochalasin 11 to prevent actin filament assembly and EGTA-AM loading to sequester calcium leaking into the chilled cells. While this combination of agents proved very effective in preserving the discoid shape of platelets after cooling, circulation studies discussed above find that these discoid platelets are still cleared at rates similar to chilled, unpreserved platelets having irregular shapes. FIG. 2 shows the clearance rates for In¹¹-labeled platelets in baboons. Each storage condition was tested in 3 animals and is expressed as the mean±SD. Platelets were labeled in vitro and then stored for 24 hr at room temperature or at 4° C. in the presence or absence of the CB and EGTA-AM preservatives. In this study, platelets chilled for 24 hr are cleared at the same rate independent of preservation and much faster than the platelets stored at room temperature. In addition to blocking the cytoskeletal rearrangements as described below, the reagent cocktail is also a potent inhibitor of phosphatidylserine exposure and of caspase activation in platelets suggesting strongly that they are not involved in the cold clearance mechanism.

Cold Clearance is Not Related to Apoptosis.

It has been suggested by some (Scherbina, A. et al., Blood 93:4222-4231; Vanagas, D. et al., Br. J. Haematol. 99:824-831; Wolf, B. et al., 94 1683-1692) but not others (Brown, S. et al., J. Biol. Chem. 275:5987-5995) that the increased clearance of stored platelets may be related to apoptotic mechanisms and that platelet undergo apoptotic-like physiological changes. For example, platelets contain caspases and during the storage of platelets, these EGTA enzymes may be activated. Platelet storage can lead to the activation of caspase 3 as judged by its hydrolysis to a lower molecular weight form. However, preincubation of platelets with the inhibitor cocktail (4° C.+CB, EGTA) completely prevents this hydrolysis despite not altering the kinetics of cold clearance. Therefore, caspase activation is not necessary for platelet removal. Similarly, we have looked carefully at the status of phosphatidylserine exposure on the outer surface of stored platelets using factor VIII binding which is considerably more sensitive than annexin binding usually employed for such studies. Such assays failed repeatedly to detect upregulation of PS during the storage platelets in the cold.

Cold-Induced Clearance Occurs in the Liver and Spleen of Mice.

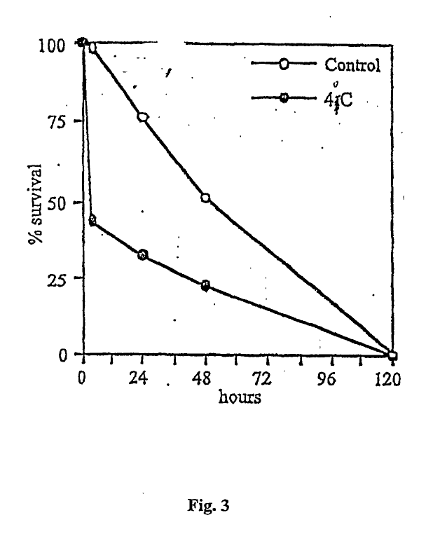

We have asked whether cooled platelets are removed by the same or different mechanisms that remove old platelets from the circulation. To simplify, we sought to identify the tissues in which platelet clearance occurs. Preliminary experiments have been preformed after loading platelets with fluorescent dyes (CMFDA) and with the radioactive marker Cr⁵¹ or In¹¹¹. Each syngeneic recipient mouse received 10⁹ platelets. After allowing the platelets to circulate for defined times in the recipient mice, various tissues were harvested and the relative uptake of platelets from the circulation determined. As shown in FIG. 3, chilled platelets (˜60% of total injected) are rapidly removed from the circulation. Platelets maintained at 37° C. circulate with a normal half time of ˜42 hrs, in good agreement with previous studies in mice (Berger, G. et al., Blood 92:4446-4452).

Platelet Clearance and Tissue Distribution.

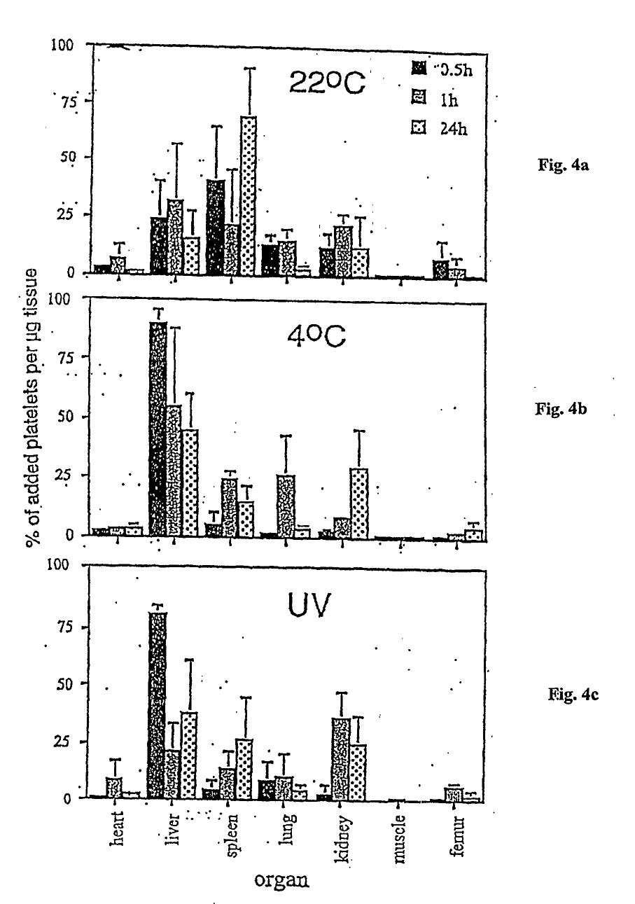

We compared the uptake in mice of In¹¹¹-labeled normal platelets maintained at 22° C., platelets chilled to 4° C. for 1 hr, and platelets activated by UV at 37° C., a condition that strongly upregulates PS to the platelet surface. Tissues were harvested 30 min, 1 and 24 hr after injection of the platelets and counted for platelet uptake. As shown in FIG. 4, liver is the primary organ where cold-treated platelets collect after their injection. The figure shows this data expressed per gram of tissue. However, in terms of total clearance, the liver contained from 85-95% of the transfused chilled platelets. Platelets remained in the tissue for >24 hr. Each time point in this experiment is the average of 4 animals ±SD. This pattern of platelet removal was not found in platelets maintained at 22° C. as the spleen in the organ in which these cells accumulate. Normal platelets circulated well for the 1 day time course of this experiment. Platelet removal after UV-activation also is highest in the liver.

Cell Type Responsible for the Uptake of Platelets in the Liver.

To determine whether the chilled platelets were bound to the endothelium in the tissues or internalized by phagocytic cells, similar experiments were done using CMFDA-loaded platelets. Spleen, liver, heart, kidney and lung were harvested 1 hr after injection of the platelets. The tissues were minced, digested with collagenase, and fluorescent cells were analyzed by flow cytometry. These experiments revealed that fluorescence was associated only with large cells (not platelet sized particles) isolated from the liver. The distribution of fluorescence in the cells derived from liver and heart was compared and demonstrated that only cells larger than platelets in liver contain platelet-based fluorescence (compare M2 zones). Very little fluorescence was found cell-associated in the other tissues from the mice. Hence, this experiment strongly suggests that the phagocytes of the liver remove chilled platelets. Since the principal phagocytes of the liver are the Kupffer cells, we postulate Kupffer cells remove the chilled platelets.

Uptake of Chilled Platelets.

The above experiments indicate that phagocytic cells mediate the removal of chilled platelets. The following experiments demonstrate that both monocytes and macrophages in vitro can phagocytize chilled platelets. Preliminary experiments were done using human monocytes isolated from blood by density gradient centrifugation as the phagocytic cell. Monocytes were fed either control platelets (37° C.) or platelets previously chilled for 1 hr at 4° C. and rewarmed just prior to their addition to the monocytes. At different times after the addition of the platelets, the monocytes were attached to coverslips and viewed in the light microscope. Light micrographs show that purified human platelets maintained at 37° C. interact minimally with monocytes in vitro. Chilled platelets, however, tightly adhere to monocytes and in many cases, appear to be ingested.

Site(s) of Platelet Clearance in Mice.

Chilled platelets are removed from the circulation by Kupffer cells in the liver. The mechanism of removal differs from the removal of senescent platelets. The results of this experiment are shown in Example 2.

Platelets are isolated from mice, labeled in some experiments with Chr⁵¹ and in others with the permeant fluorophore 5-chloromethyl fluorescein diacetate (CMFDA: Molecular Probles, Inc.), washed and reintroduced into syngeneic mice. Before injection into the mice, labeled platelets will be: (a) maintained at 37° C.; (b) chilled to 4° C. for 1 hr; (c) activated with 1 U/ml of thrombin to induce P-selectin upregulation; (d) activated with 1 μM of the ionophore A23187 to induce phosphatidylserine upregulation; and (e) conditions (a) and (b) in the presence of inhibitors of shape change (cytochalasin B, EGTA, taxol). The preservatives are used because they eliminate certain factors such as caspase activation and PS expression. ˜10⁸ platelets are introduced into each animal.

To determine the location of platelet uptake, mice are sacrificed at 0, 2, 6, 24, and 72 hrs. The weight of each animal is recorded before sacrificing and the animals are bled. Liver, spleen, heart, lungs, kidneys, skeletal muscle, and the femurs are removed from each animal. The % of transfused platelets is determined by counts (cpm) of platelet rich plasma versus platelet poor plasma (control for leakage out of platelets). The weight of each organ is determined and the total cpm count per organ determined. Heart and skeletal muscle serve as controls for trapped blood volume in the organs. If necessary, the animal and organs are perfused prior to isolation.

These experiments allow us to confirm and more precisely map the locations of platelet uptake in mice after chilling, as it normally occurs as platelets age and after robust PS expression by UV treated platelets. Once these data are in hand, experiments will shift to experiments injecting fluorescently labeled platelets with the goal of confirming the cell types from each tissue identified above that contain ingested platelets.

Identification of Cell Type.

Transfusion experiments are repeated using CMFDA-loaded platelets. In these experiments, as in those above, the survival times of the circulating platelets are followed. Blood is removed at increasing times after injection of labeled-platelets and the amount of fluorescent platelets remaining at each time compared to the starting values. Organs and tissues are harvested and divided. Half of each organ/tissue is fixed with 4% paraformaldehyde, frozen, thin sections prepared, and the location of platelets determined in a fluorescence microscope. Sections are counterstained for platelets (P-selectin, GPIIb/IIIa) and macrophage antigens. The remaining organ pieces are chopped, digested and collagenase, and platelet-specific fluorescence analyzed by flow cytometry (FACS) of the cell suspension generated by this procedure. The large cells containing platelets are identified by counterstaining with cell specific antibodies, i.e., anti-CD11b and CD14 for macrophages, anti-CD31 for endothelial cells, etc. We also label with anti-α_{IIb}β₃ to demonstrate that the platelets are internalized and not bound to the surface of the cells identified by FACS. A very similar FACS assay has recently been used to quantitative phagocytosis of aged and dying platelets. (Brown, S. et al., J. Biol. Chem. 275:5987-5995).

These experiments, combined with those above, are used to confirm the identification of the organ and cell type within the organ that is responsible for normal platelet clearance, platelet clearance after chilling, and platelet clearance once PS is exposed on their surfaces. We assume, based historically and on our preliminary data, that the cells having ingested platelets are macrophages.

Cold Protective Procedures Alter the Pattern of Platelet Clearance.

Recent efforts have focussed on agents that can prevent membrane phase transitions induced by cooling such as simple sugar compounds such as trehalose. The following experiments determine the extent to which platelets remain in the circulation of mice when chilled in the presence of increasing concentrations of trehalose and the effect of storage time in this compound in the cold on clearance.

Development of a Quantitative Assays for Phagocytosis of Cold-Treated Platelets.

Macrophages isolated from the tissue identified above, and maintained in culture, can be specifically induced to ingest chilled platelets.

We developed a quantitative assay for cold-platelet phagocytosis and used it to identify the macrophage receptor(s) that lead to recognition and ingestion of cold-treated platelets. A specific example of this assay is provided in Example 2. In general, phagocytic assays use monocytes isolated from human blood, mouse peritoneal macrophages, and tissue macrophages isolated from the organ(s) identified above, e.g. Kupffer cells from the liver, etc. Platelets are loaded with 5 μM CMFDA at 37° C. for 30 min, washed by centrifugation in buffers containing PGE₁ and then: (a) chilled for 1 hr; (b) activated with 1U/ml of thrombin for 5 min; or (c) activated with the 1 μM A23 187 ionophore. Platelets are mixed with macrophages/monocytes at ˜10-20 to 1 ratios and incubated for different time at 37° C. (2-30 min). Free platelets are separated from those associated with macrophages by differential centrifugation at speeds that pellet macrophages but not platelets. Cells are washed twice and then fixed 4% paraformaldehyde in PBS. Fixed cells are incubated with FITC or TRITC-tagged anti-platelet GPIbα or αIIbβ3 antibodies. The purpose of the anti-platelet antibodies are to dissect internalized from surface adherent platelets. Bound platelets are double labeled by this approach, e.g., fluorescence from the CMFDA-loading procedure and counter fluorescent-labeled by a second fluorophone attached to the anti-platelet receptor antibody. Internalized platelets fluoresce only at emission wavelength of the loaded CMFDA. Cells are examined in the fluorescence microscope after attaching them to coverslips. Platelet uptake by monocytes/macrophages are quantified by flow cytometry.

To definitively demonstrate specificity for the cold reaction, we use P-selectin −/− platelets in some of the phagocytic assays. P-selectin has been shown to mediate the adherence of activated platelets to leukocytes. However, since cold-induced platelet clearance still occurs with similar kinetics in P-selectin −/− mice, P-selectin should not be required for platelet ingestion by macrophages after chilling. P-selectin −/− cells are chilled and fed to macrophages. The phagocytic rate should be similar to chilled platelets from wild-type mice.

Cold-induced phagocytosis is regarded as specific when cold-exposed platelets are ingested but control cells, maintained in the warm and thrombin-activated cells are not. We believe that ionophore treated cells also are ingested but by a different mechanism from the cold-treated cells.

Confirmation of the Identification of the Macrophage Receptor that Recognizes Chilled Platelets.

There exists a specific receptor(s) on Kupffer cells that binds to and initiates phagocytosis of platelets exposed to the cold.

Professional phagocytes use multiple receptors find and ingest particles. Many of these receptors are well characterized and anti-receptor reagents are widely available and in a number of cases, knockout mice lacking these receptors have been produced. Specify inhibitory and/or competitive reagents are added to the phagocytic assays in attempts to identify the pathway for uptake of chilled platelets. Once a candidate receptor has been identified in the phagocytic assays in vitro, knockout mice are used to confirm that chilled platelets have increased circulatory times in mice whose phagocytes lack this receptor. Table I lists possible macrophage and platelet receptors and reagents available for these studies. The experiments which confirmed that αmβ2 is a macrophage receptor that recognizes chilled platelets is described in Example 2.

| | | Reagents | Possible | |
|---|---|---|---|---|
| | | available for | platelet | |
| Macrophage | Knockout | inhibitory | receptor | |
| receptor | mice | studies | partner | References |
| FcR family | Available- | IgGs | IgG bound | (Indik, Z. |
| FcγRI | Ravitch | | to platelet | et al., |
| FcγRIIA | and | | FcRIIA | Blood |
| | Schrieber | | | 86: 4389- |
| FcγRIII | (Indik, | | | 4399) |
| (CD16) | Ibid.) | | | |
| CRs (C3b, | Available | Anti- | vWfR - this | (Caroll, M., |
| C3bi) | | CD11/CD18 | interaction | Annu. Rev. |
| CR1 | | Anti-GPIb | can be | Immunol. |
| CR2 | | (mice | inhibited | 16: 421- |
| CR3 | | and human) | using | 432) |
| (αMβ2: | | Mice lacking | mocarhagin. | |
| CD11b/ | | GPIb or Mac 1 | | |
| CD18) | | EGTA | | |
| CR4 | | RGDS | | |
| (αxβ2) | | RGES | | |
| Mannose | | Mannose | | (Ezekowitz, |
| | | lectins | | R. et al., |
| | | | | J. Exp. |
| | | | | Med. |
| | | | | 172: 1785- |
| | | | | 1794) |
| | | | | (Taylor, |
| | | | | M., et al., |
| | | | | J. Biol. |
| | | | | Chem. |
| | | | | 265: 12156- |
| | | | | 12162) |
| Class A | | AcLDL | | (Platt, N. et |
| Scavenger | | Fucoidan, | | al., Proc. |
| SR-A | | Poly- | | Natl. Acad. |
| (acetylated | | inonsitol | | Sci., USA |
| LDL) | | anti- | | 93: 12456- |
| | | scavenger | | 12460) |
| | | receptor IgG | | |
| | | mAb 2FS | | |
| Class B | | Phospho-L- | PS | (Savill, J. |
| Scavenger | | serine | | et al., J. |
| CD36 | | Phosphatiyl- | | Clin. |
| | | serine | | Invest. |
| | | | | 90: 1513- |
| | | | | 1522) |
| | | | | (Navazo, |
| | | | | M. et al., J. |
| | | | | Biol. |
| | | | | Chem. |
| | | | | 271: 15381- |
| | | | | 15385) |
| PS receptor | | vesicles | | |
| | | Anti-CD36 | | |
| | | receptor | | |
| | | (monoclonal | | |
| | | 217) | | |
| CD14 | | | | (Fadok, V. |
| | | | | et al., |
| | | | | Nature |
| | | | | 405: 85-90) |
| PECAM- 1 | | | PECAM-1 | (Sun, Q. -H. |
| | | | | et al., J. |
| | | | | Biol. |
| | | | | Chem. |
| | | | | 271: 11090- |
| | | | | 11098) |
| Vitronectin | | | PECAM-1 | (Savill, J. |
| (αvβ3) | | | | et al., |
| | | | | Nature |
| | | | | 343: 505- |
| | | | | 509) (Piali, |
| | | | | L., et al., J. |
| | | | | Cell Biol. |
| | | | | 130: 451- |
| | | | | 460) |
| SIRPα | | Anti-CD47 | CD47 | (Oldenborg, |
| | | SIRPa | | P. -A. et |
| | | knockout | | al., Science |
| | | mice | | 288: 2051- |
| | | | | 2054) |

Mac1-vWfR.

We first block the phagocytosis of chilled platelets with specific anti-phagocytic receptor IgGs. Macrophage cultures are incubated with anti αMβ2 antibodies (alternatively referred to herein as anti Mac-1 antibodies (CD11b/CD18)) at 4° C. and unbound IgG removed by washing. Chilled platelets preloaded with fluorescent dye or control platelets maintained in the warm are added, and the number of platelets ingested per macrophages determined with time incubation determined. If significant inhibition of phagocytosis is measured (30-70% decrease), we shift to Mac-1 −/− animals for more definitive proof.

Experiments use macrophages isolated from these animals as well as use the animals for classic circulation studies. Macrophages are isolated from the liver and the peritoneal cavity of knockout and control matched background wild-type mice. The ability of these two populations of macrophages (−/− and +/+ Mac-1) to phagocytize chilled platelets are compared. If knockout mice display diminished phagocytosis of the chilled platelets this finding is confirmed in circulation studies in these animals.

We continue to work through the above list of phagocytic receptors to identify one or more receptors involved in this process. See Example 2 for confirmatory evidence of the role played by αmβ2 in the clearance of chilled platelets.

Identification of Alterations at the Platelet Surface that Lead to Uptake and Aggregation.

There are two possible alterations that could occur at the surface of the chilled platelet to elicit clearance. Hypothesis 1 is a conformational change, or aggregation, of one or more glycoproteins present on the surface of the resting cell leads to recognition and ingestion by macrophages. Hypothesis 2 is that cold induces either the lost, or appearance, of surface molecules required to prevent or cause phagocytosis.

We take a similar approach to identify the platelet surface molecules that mediate phagocytosis as was used above to identify the macrophage receptor that mediates platelet clearance. Antibodies against platelet surface proteins are issued in attempts to block clearance and when possible, platelets are obtained from knockout mice lacking specific receptors and used in the cold-induced phagocytic assays in vitro and in animal circulation studies. Once the identity of the molecule(s) involved in this reaction is confirmed, we determine how cold leads to altered function (i.e., removal, appearance, change in conformation, or change in surface aggregation). See Example 2 for confirmatory evidence of the role played by platelet GPIb in the clearance of chilled platelets.

Determination of the Extent to which Altered Receptor Function Leads to Clearance.

We use anti-human platelet receptor antibodies in attempts to block cold-induced phagocytosis. Antibodies against all major, and most of the minor, human platelet surface receptors are widely available. We first determine the extent to which combinations of these antibodies when added to platelets during the cold treatment process block phagocytosis. If bound antibody induces phagocytosis, we use Fabs instead of intact IgG. Exemplary antibodies are described in Example 2.

We use platelets from various knockout mice to search for platelets that are not ingested after cooling. Animals available include: (a) vWfR lacking mice (GPIbα knockout (Ware, J. et al., Proc. Natl. Acad. Sci., USA 97:2803-2808); (b) CD36 −/−; (c) P-selectin −/−; (d) CD47 −/−; (e) α_{Πb}β₃; and (f) others. Platelets are harvested from mice, labeled with CMFDA, divided into three groups -control, chilled, and activated. Labeled platelets are incubated with macrophages for increasing lengths of time after which the amounts of ingested platelets are quantified. Exemplary knockout mice are described in Example 2.

Following the identification of molecules responsible for initiating phagocytosis, we determine how their function is altered by cold. For example, if the vWfR receptor on the surface of cold platelets now triggers phagocytosis through engagement of CD11b/CD18 on macrophages, we determine if this finding results from altered function of the vWf receptor or to its aggregation on the surface of the chilled platelets. Alterations in glycoprotein function could be identified by differential binding of monoclonal antibodies to GPIb or other proteins on resting versus chilled platelets. Differences in aggregation are investigated in the electron microscope after labeling with anti-GPIba IgG-coated gold particles. Platelets are fixed, labeled with antibody-colloidal gold complexes, and the surface distribution of the antibody-gold complex mapped in the electron microscope after rapid freezing and freeze-drying of the platelets.

These experiments are useful for identifying proteins expressed normally on the platelet surface whose function is altered to initiate phagocytosis. The utility of the knockout animals is to confirm that the receptor identified in the in vitro experiments is indeed necessary for the removal of chilled platelets from the circulation.

Additionally or alternatively, the signal for phagocytosis requires the loss of a protein from the surface of the chilled platelet. Such a critical molecule could be a known surface glycoprotein as CD47. Molecules lost after chilling are further characterized by specifically blocking the protein function using antibodies and determining if blockage leads to phagocytosis of platelets maintained at 37° C. Thereafter, we define whether such platelets are cleared from the circulation of mice.

Cooling Promotes Platelet Aggregation via vWfR which Leads to Clearance after Transfusion.

The membrane phase transition induced by cooling increases the avidity of the vWfR for vWf. This interaction leads not only to platelet-platelet aggregates but to platelet-leukocyte aggregates.

We determine the extent to which vWfR (GPIb/IX/V complex) mediates cold-induced aggregation. First, we determine if RGD and/or function blocking α_{Πb}β₃ antibodies affect this aggregation reaction to eliminate the fibrinogen receptor from this pathway. We also use platelets from Glanzman's patients to determine if they have the reaction. Next, we determine the extent to which vWfR is the key molecule by using antibodies that block the binding of vWf to platelets. Similarly, platelets from mice lacking vWfR can be used. Finally, we determine the extent to which leukocytes are a component of these aggregates.

Elimination of αΠbβ3 from this aggregation process. Activated platelets aggregate primarily because of the up-regulation and increased activity of αΠbβ3. Activated αΠbβ3 forms bridges via fibrinogen/fibronectin/fibrin (RGD-containing proteins) in plasma. However, allegedly chilling does not up-regulate 2b3a or P-selectin (which could bind PSLG on platelets). We first confirm that πΠbβ3 is not involved. We block the function of αΠbβ3 using 300 MRDGS peptide added to PRP; or by using platelets from Glanzman patients. If aggregation is induced by cold under these conditions, we focus on the role of the vWfR in this process.

Exploration of the Function of vWfR in this Aggregation Process.

We first define if the aggregation is mediated by vWf-vWfR. Antibodies that block that vWf binding site on GPIbα are used. If these experiments prohibit cold-induced aggregation, we determine the extent to which cold promotes the binding of purified vWf and A1 domain to platelets and the extent to which there is sufficient vWf inside platelets to promote aggregation in the cold in gel-filtered platelets, i.e., without PRP and without added vWf. Platelet binding to vWf is assessed by first coating surfaces with vWf or its Al domain that then comparing the attachment of platelets maintained at 37° C. to platelets cooled to 4° C. to these surfaces.

Human and Animal Subjects:

Blood is obtained from normal healthy human subject volunteers over the age of 18 years. All donors will have hematocrits of ˜40. We anticipate that approximately 100 donor samples of 10 to 40 ml are used each year. Blood is used to isolate normal human platelets and these cells are used to answer basic questions on the structure of platelet actin-cytoskeleton function. These are not clinical studies. Blood from other sources cannot be used because of the unique structure of the human blood platelet.

Transgenic mice lacking WASP, gelsolin, and other potential regulatory proteins as they become available are maintained. We use approximately 400 mice/year for the experiments, and plan to have an average residence population of 200. Both males and females are used for the experiments in balanced numbers, and animals are used at age 6-8 weeks. Mice are being used because they are the only mammalian species that is tractable for gene-knockout techniques. They also represent the closest, easily manipulable equivalent for analysis of mammalian physiology with relevance to humans.

### Example 2

### Characterization of Platelet Clearance

This example reports the results of several experiments described in Example 1.

We developed the capacity to measure platelet clearance and distribution in mice. Platelets were removed from normal mice or mice engineered to lack expression of specific platelet or macrophage receptors, labeled with a fluorescent dye or else with radioactive indium and then injected into syngeneic animals. Depending on the experiment, persistence of platelets in the circulation, tissue distribution or the behavior of platelets in vascular beds was measured. The results we have obtained are described in summary form below. More detailed descriptions of the experimental protocols and tabular and graphic renditions of the experimental findings also are presented below.

Mouse platelets kept at room temperature have a circulation half time of about 40 hours, as previously reported by others (Berger, G. et al. Blood. 92:4446, 1998). Consistent with older studies, room-temperature platelets accumulated primarily in the spleen as well as in the liver with time. As observed in humans, over half of platelets chilled for one hour are rapidly cleared (Becker, G. et al. Transfusion. 34:61, 1973), and, in contrast to the distribution of room-temperature platelets, the rapidly disappearing population of chilled platelets almost entirely went to the liver. The remnant population clears at about the same rate as room temperature-treated platelets. Real-time intravital microscopy of the liver circulation showed that cleared chilled platelets co-localize with phagocytic scavenger cells (macrophages called Kupffer cells) in the peripheral hepatic sinuosoids. These results indicated that cooling induces changes in the platelet leading to recognition by liver macrophages.

The major constitutively expressed membrane receptor on platelets in the von Willebrand factor receptor, a complex of glycoproteins designated GPIb/V/IX (GPIb).

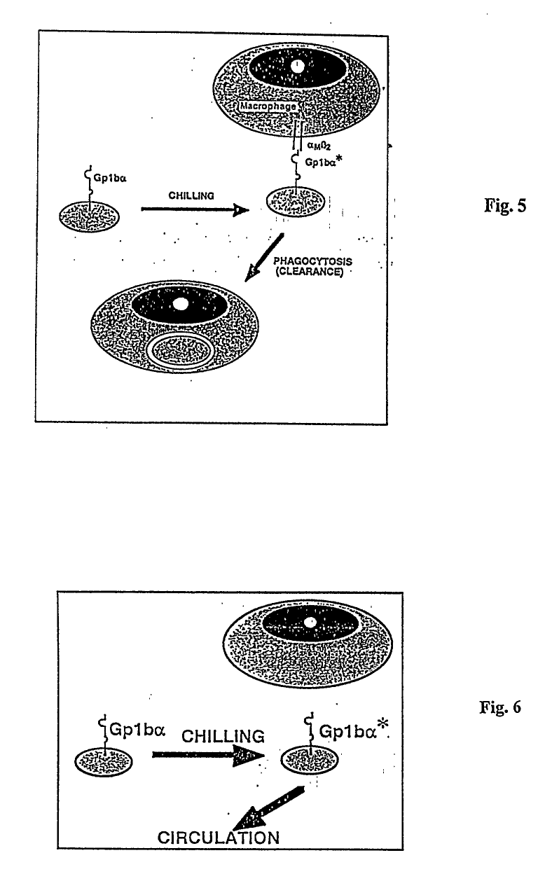

Recent work has shown that GP1b binds a major macrophage phagocytic receptor αMβ2 integrin. Therefore a change in GP1b induced by cooling (indicated by an asterisk * in FIG. 5) seemed a good candidate on the platelet side becoming recognized by macrophage αMβ2 integrin receptors.

To test this hypothesis, we infused chilled platelets into normal and syngeneic mice lacking αMβ2 integrin receptors, and the circulation of these platelets was indistinguishable from room-temperature platelets. In addition, chilled platelets did not show the increased adherence to hepatic αMβ2 integrin knockout macrophages seen in wildtype macrophages (FIG. 6).

The normal circulation of chilled platelets in αMβ2 integrin knockout mice suggests that other macrophage receptors may not play as significant a role as GPIb in the clearance of the platelets. For example, ultraviolet (UV) radiation of platelets causes changes associated with programmed cell death (apoptosis) including external expression of phosphatidylserine (PS). PS-exposure leads to clearance of cells by specific macrophage receptors, but these receptors do not seem to be significantly involved in the removal of cold platelets. Consistent with this conclusion, we find no increased expression on chilled platelets treated with cytochalasin B and EGTA-AM, despite the fact that these platelets clear rapidly after cooling.

To further assess the role of platelet GPIb in cold-mediated clearance, this glycoprotein was removed from the external surface of human platelets with the proteolytic enzyme mocarhagin, and the platelets were infused into mice. Human platelets were used because mocarhagin does not work on mouse GPIb. Although human platelets clear rapidly from mouse circulations, the retention of enzyme-treated platelets by hepatic macrophages was 3-4 times less than of untreated platelets.

In conclusion, chilling, possibly by eliciting lipid membrane phase transitions, induces a conformation change in the extracellar domain of platelet GPIb that causes its recognition by macrophage αMβ2 integrins. A modification of these interactions is the basis of our current technology for preventing cold-induced platelet clearance.

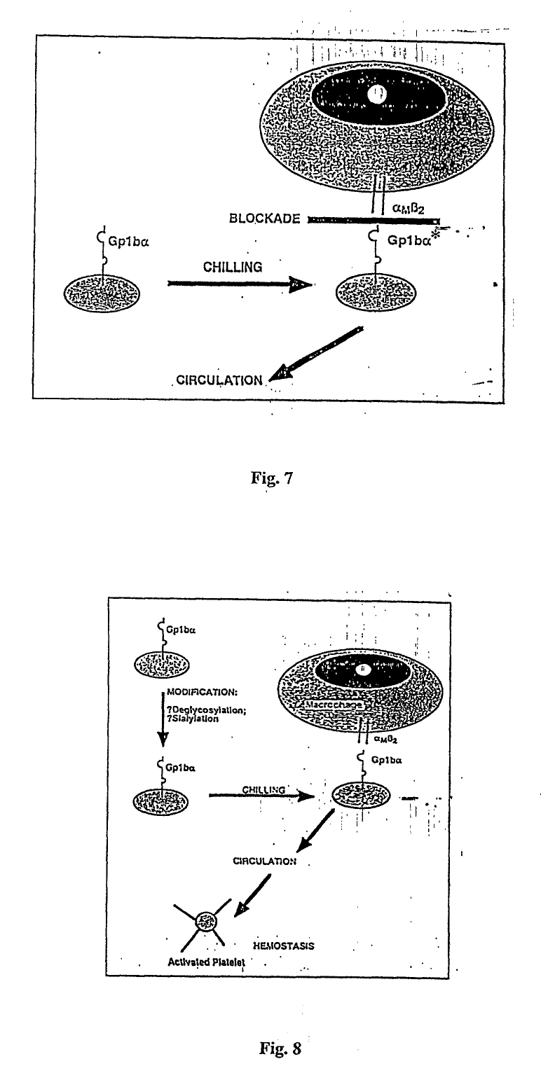

In theory, identification of the specific domains involved in platelet GPIb-αMβ2 integrin binding and development of inhibitors diminishing the affinity of this interaction should permit chilled and rewarmed platelets to circulate normally (FIG. 7). Monoclonal antibodies selective for these binding proteins, peptides or small molecule inhibitors should be considered as blocking agents, and the inhibition could be directed at either the platelet or the macrophage.

αMβ2 integrins are not only expressed on macrophages throughout the host, but they are also important host-defense receptors Therefore, blockading these molecules is not as attractive, especially since platelet recipients would have to receive a systemic treatment, and many platelet transfusions are given to immunosuppressed patients. To identify reagents that bind the platelet GPIb with sufficiently high avidity and that could be administered ex vivo after platelet procurement for transfusion is a more appealing approach, provided that these agents do not significantly impair the interaction of platelet GPIb with targets important for hemostasis such as von Willebrand factor.

An alternative approach would be to remove part of GPIb from platelets ex vivo, so as to render platelets that do not bind aMP2 integrins after chilling but retain hemostatic capability (FIG. 8). By this approach, no exogenous chemicals would require evaluation in patients and the toxicity evaluation would be limited to the treated platelets themselves. We believe that the “part” most amenable to removal without inactivating GPIb may be selected sugars. Selected sugars are implicated in the interactions between αMβ2 integrins and some of their targets (Thornton, B. et al., Transfusion. 34:61, 1973; Thornton, B., et al., J. Immunol. 153:1769, 1996), and erythrocytes can be modified by removal of immunogenic sugars and circulate normally (Kruskall, M. et al., Transfusion. 40:1290, 2000).

As a first step toward evaluation of this strategy, we have developed an in vitro phagocytic assay to study the interaction between chilled platelets and mononuclear phagocytes. We first showed that cooled, but no UV-treated or thrombin-activated platelets adhere to and are ingested by cultured human monocytes. To increase convenience of the assay, we used a human monocytic cell line, THP1 cells and showed that after activation, these cells also bind cooled platelets. These measurements can be made with a fluorescence-activated cell sorter (FACS). Using the assay, we showed that relatively low concentrations (>50 mM) of the sugars, α-methyl-D-mannoside, α-methyl-D-glycoside, and β-methyl-D-glycoside but not N-acetyl-D-glucosamine, D-glucose or D-galactose (up to 200 mM) inhibited phagocytosis of platelets by phagocytic cells. None of these sugars had any effect on platelet aggregation induced by thrombin or by ristocetin. The latter result is particularly important, because it reflects platelet hemostatic function mediated by GPIb interaction with von Willebrand factor.

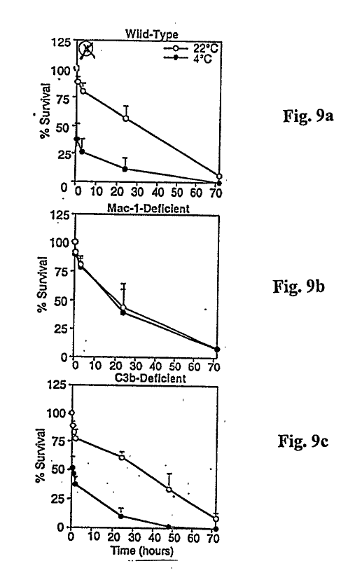

The in vitro phagocytic assay appears to be one reasonable approach to screen agents that may be platelet clearance antagonists. An alternative approach is to use several of the numerous monoclonal antibodies to epitopes on GPIb. If one or more of these antibodies can be shown to have altered reactivity with room temperature versus chilled platelets, its reaction might correlate with the GPIb conformational change associated with chilling, and a comparable variation in reactivity might report successful removal of the sugar or sugars involved in GPIb recognition by αMβ2 integrin (FIG. 9).

Materials and Methods.

Mouse livers are prepared for intravital microscopy and CMFDA-loaded platelets injected after maintenance in the warm or chilling to 4° C. for 1 hr into the jugularis vein. Platelet clearance was followed for 30-90 min. Phagocytic cells (Kupffer cells) were identified by the injection of 0.1 μm opsonized-latex particles (appeared red) marked with a nile-red-fluorescent dye. Blood enters the lobules from the portal vein and hepatic arteries, flows through the liver sinusoids and then exits via the central vein. Kupffer cells reside primarily at the periphery of the liver sinusoids and it is this peripheral region where cooled-platelets become trapped. The results show the distribution of phagocytes (appeared red) and platelets (appeared green). Co-staining of platelets and macrophages appeared yellow. The lobule organization of the liver was indicated (CV, central vein). These studies reveal our ability to visualize platelet removal in real time in living animals.

Platelet Clearance After Cooling.

Cold-induced platelet clearance occurs predominantly in the liver and spleen of mice. Approximately 60% of the injected chilled platelets are rapidly removed from the circulation of mice in a similar fashion to cold platelets in primates. Mouse platelets maintained at 22° C. have circulation half time of ˜42 hrs, in keeping with previous studies. To determine the sites of platelet clearance, 4° C., 22° C.- and UV-treated platelets, loaded with ^{1 1}Indium, were injected into syngeneic mice and the incorporation of radioactive label into organs and tissues determined.

Exposure of platelets to UV is known to upregulate phosphatidylserine to the platelet surface, a condition that leads to their rapid removal. Tissues were harvested at 30 min, 1 hr and 24 hr after platelet injection and the relative uptake of platelets from the circulation determined. The liver is the primary organ where cold-treated platelets collect. In terms of total clearance, the liver contained 85-95% of the transfused chilled platelets after 24 h. These data are expressed per gram of tissue. Each time point in this experiment is the average of 4 animals ±SD. This pattern of platelet removal was not found in platelets maintained at 22° C. where the spleen is the primary organ in which these cells accumulate. 22° C. maintained platelets circulated normally for the 1 day time course of this experiment. The liver was also the primary site of platelet removal after UV-treatment. We confirmed these results transfusing CMFDA (a fluorescent dye)-loaded platelets into mice, isolating cells from the liver and demonstrating the incorporation of fluorescent label into these cells by FACS analysis. Hence, these experiments strongly suggest that the phagocytes of the liver recognize and remove chilled platelets. Since the principal phagocytes of the liver are Kupffer cells, we postulated the Kupffer cells bind and remove the chilled platelets.

Cooled Platelets Circulate in αMβ2-integrin deficient mice, but not in C3 deficient mice. FIG. 9 shows that chilled wild type mouse platelets circulate with the same half-life time as platelets stored at room temperature in αMβ2-integrin deficient animals, but not in C3-deficient mice. Platelets were loaded with CMFDA and each recipient mouse received 108 platelets. Platelets chilled for 1 hour and platelets stored at room temperature were transfused into wild type (WT), αMβ2-integrin-deficient, or C3-deficient mice. Blood samples were taken immediately and at 0.5, 2, 24 and 72 hours after the platelet infusion.

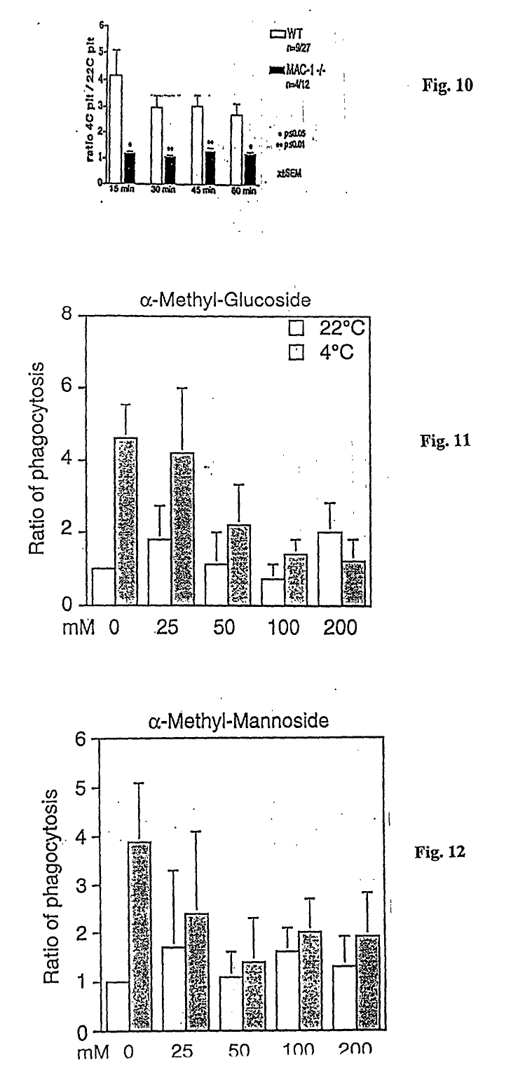

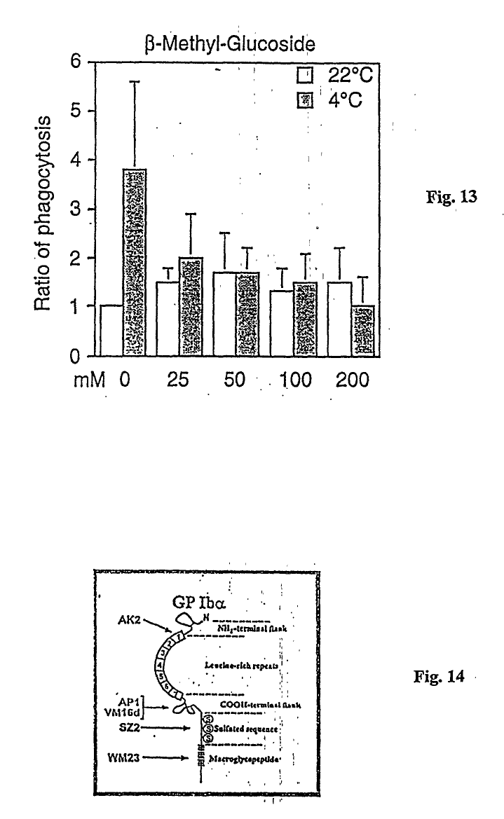

FIG. 10 shows that αMβ2-integrin deficient liver-phagocytes also fail to bind chilled platelets. Mouse livers were prepared for intravital microscopy as described and a mix of 108 each of chilled CMFDA- or room temperature stored TRITC-labeled platelets was injected into the jugularis vein of recipient WT or αMβ2-integrin deficient mice. The ratio of red to green was determined with time from video frames and was plotted. Platelet clearance was followed for 90 min. Cold treated platelets were avidly bound by Kupffer cells in the WT mouse (3:1 ratio of cold to warm platelets adherent to liver sinusoids) but not by the αMβ2-integrin deficient Kupffer cells (1:1 ratio of cold to warm, i.e., equal binding). The results clearly demonstrate that the αMβ2-integrin is the receptor on liver macrophages leading to their phagocytosis.

In vitro Assay for the Phagocytosis of Chilled Platelets.

The experiments above have shown that Kupffer cells remove chilled platelets through the αMβ2-integrin. The following experiments demonstrate that human monocytes also selectively phagocytize chilled human platelets. Preliminary experiments used human monocytes isolated from blood by density gradient centrifugation. Monocytes were incubated for 1 hour at 22° C. with either control platelets (22° C.) or platelets previously chilled for 1 hr at 4° C. and rewarmed just prior to their addition to the monocytes. Human platelets maintained at 22° C. interact minimally with monocytes in vitro but after chilling, many platelets tightly adhere to and appear to be ingested by the monocytes.

THP-1 Monocytic Cells also Selectively Phagocytize Chilled Human Platelets, and Flow Cytometry can be Used to Measure Platelet Phagocytosis.

In these experiments (FIG. 11, 12, and 13) platelets were loaded with fluorescent dye CM-Orange, then incubated for 1 h at 22° C. (control), 4° C. (chilled) or exposed to UV (UV). Phagocytosis was quantified by measuring the incorporation of CM-orange-fluorescence (CM-Orange labeled platelets) into monocyte-sized cells. Bound versus ingested platelets were separated by labeling the cells after the phagocytic period with FITC-labeled anti-integrin-β3 (CD 61) antibodies, e.g., bound platelets will be FITC positive, ingested platelets will be negative. The uptake of control versus chilled platelets was compared. Chilled platelets are phagocytized because they fail to label with FITC-conjugated anti-β3 antibodies (CM-Orange positive), whereas control platelets are poorly ingested although some platelets are bound to the monocyte surface. From this experiment, the ratio of 22° C. to 4° C. ingested platelets (CM-Orange positive) can be calculated. The results from 3 experiments (mean ±SD) show that cold platelets were ingested 3-times more effectively than control platelets. Surprisingly, UV treated platelets, or platelets activated with 1U/ml thrombin for 5 min, were poorly ingested (sarne ratio as platelets kept at 22° C.). These findings indicate that the cold platelet clearance differs from UV-induced clearance.

The a chain of the vWfR has a binding site for the αMβ2-integrin. We studied the adherence of cold-treated human platelets in liver sinusoids of wild type mice using intravital microscopy, and compared the adherence-ratio of sham- and mocarhagin- (a snake metalloproteinase, specifically cleaving the N-terminus of human GPIbα) treated platelets. These studies indicate that cold platelets adhere 3-4 times more to sinusoids than the same platelets having GPIbα removed. Hence, we believe that the αMβ2-GPIbα receptor pair clears chilled platelets. This observation makes αMβ2-GPIbα a particularly attractive receptor pair. First, the avidity of GPIbα can be modulated by the underlying cytoskeleton, providing a mechanism to transfer cold induced cytoskeletal rearrangements to the platelet surface. Cold may also promote vWf binding and potentiate clearance. Second, coldper se does not cause the removal of vWfR from the membrane surface, while activation of cells with thrombin at 37° C. does, and such activated platelets are not cleared. This suggests that activation by thrombin of cold-treated platelets after rewarming might enhance their circulation and that agents which prevent the down regulation of vWfR might cause thrombin-activated platelets to be cleared.

To confirm the identity of vWfR as an important counterreceptor, we first block the phagocytosis of chilled platelets with specific anti-GPIb-antibodies. FIG. 14 shows the epitope map of monoclonal antibodies to GPIbα, schematically represented on the extracellular domain of GPIbα. The following monoclonal antibodies are employed: AK2 (binds within the first leucine-rich repeat [amino acid residues 36-58]); AP1 and VM16d (bind to the COOH-terminal flanking and leucine-rich repeat region (201-268); SZ2 (maps to the sulfated tyrosine residues encompassing amino acids 268-281); WM23 (binds within the macroglycopeptide region of GPIbα). Alterations in glycoprotein function can be identified by differential binding of monoclonal antibodies to GPIba, binding of vWF or other proteins on resting versus chilled platelets. Furthermore, we use the A1 domain of vWf, glycocalicin and the I-domain or the lectin-binding domain of the αMβ2-integrin to inhibit the interaction of vWfR and the αMβ2-integrin. To eliminate phagocytosis induced by the Fc domain of bound IgGs, we will prepare F(ab)2's and use them as blocking agents. Unbound IgGs and other inhibitors are removed by washing. As a second approach, GPIbα is cleaved from the surface of the human platelet using mocarhagin. Chilled platelets, preloaded with fluorescent dye or control platelets maintained in the warm is added to macrophages and the number of platelets ingested per macrophage determined with time of incubation. If significant inhibition of phagocytosis is measured (30-70% decrease), we isolate platelets from GPIbα-deficient animals, and perform phagocytic assays and classic circulation studies to confirm that cooled cells are still cleared.

Each of the references, patents and patent publications identified or cited herein is incorporated, in its entirety, by reference.

Although this invention has been described with respect to specific embodiments, the details of these embodiments are not to be construed as limitations. Various equivalents, changes and modifications may be made without departing from the spirit and scope of this invention, and it is understood that such equivalent embodiments are part of this invention.

We claim:

1. A method for identifying a platelet clearance antagonist, comprising:
Contacting a chilled platelet with a liver macrophage in the presence and in the absence of a test molecule; and
Detecting binding of the chilled platelet to the liver macrophage,
Wherein a reduction in the binding in the presence of the test molecule relative to the binding in the absence of the test molecule indicates that the test molecule is a platelet clearance antagonist.

2. A method for identifying a platelet clearance antagonist, comprising:
Contacting an isolated platelet ligand with a liver macrophage in the presence and in the absence of a test molecule; and
detecting binding of the platelet ligand to the liver macrophage, wherein a reduction in the binding in the presence of the test molecule relative to the binding in the absence of the test molecule indicates that the test molecule is a platelet clearance antagonist.

3. A method for identifying a platelet clearance antagonist, comprising:
contacting an isolated platelet ligand with an isolated liver macrophage receptor in the presence and in the absence of a test molecule; and
detecting binding of the platelet ligand to the liver macrophage receptor, wherein a reduction in the binding in the presence of the test molecule relative to the binding in the absence of the test molecule indicates that the test molecule is a platelet clearance antagonist.

4. A method for identifying a platelet clearance antagonist, comprising:
Contacting a chilled platelet with an isolated liver macrophage receptor in the presence and in the absence of a test molecule; and
detecting binding of the chilled platelet to the liver macrophage receptor, wherein a reduction in the binding in the presence of the test molecule relative to the binding in the absence of the test molecule indicates that the test molecule is a platelet clearance antagonist.

5. The method of any of claims 1-4, inclusive, wherein the platelet clearance antagonist is a platelet antagonist.

6. The method of claim 5, wherein the platelet antagonist binds to a platelet ligand selected from the group of platelet ligands provided in Table 1.

7. The method of claim 5, wherein the platelet antagonist binds to a platelet ligand that is vWfR or a subunit thereof.

8. The method of any of claims 1-4, inclusive, wherein the platelet clearance antagonist is a liver macrophage receptor antagonist.

9. The method of claim 8, wherein the liver macrophage receptor is a Kuppfer cell receptor antagonist.

10. The method of claim 8, wherein the liver macrophage receptor antagonist binds to a liver macrophage receptor selected from the group of liver macrophage receptors provided in Table 1.

11. The method of claim 8, wherein the liver macrophage receptor antagonist binds to a liver macrophage receptor that is αMβ2.

12. The method of claim 1, wherein detecting binding of the platelet to the liver macrophage comprises detecting phagocytosis of the platelet by the macrophage.

13. A method for preparing a platelet antagonist-treated platelet for transfusion, comprising:
Contacting a chilled platelet with a platelet antagonist under conditions to permit the chilled platelet antagonist to bind to a platelet ligand on the chilled platelet and, thereby, form a platelet antagonist-treated platelet.

14. The method of claim 13, wherein the platelet antagonist selectively binds to a platelet ligand identified in Table 1.

15. The method of claim 13, wherein the platelet antagonist selectively binds to vWfR or a subunit thereof.

16. The method of claim 13, 14, or 15, wherein the platelet antagonist is an antibody or antibody fragment.

17. The method of claim 13, wherein contacting is performed after the platelets are chilled.

18. The method of claim 13, wherein contacting is performed in a platelet bag.

19. The method of claim 13, wherein the chilled platelets are treated with at least one of a first agent for inhibiting actin filament severing and a second agent for inhibiting actin polymerization.

20. The method of claim 19, wherein the chilled platelets are treated with a first agent for inhibiting actin filament severing.

21. The method of claim 19, wherein the first agent is an intracellular calcium chelator.

22. The method of claim 21, wherein the intracellular calcium chelator is selected from the group consisting of: QUIN, STIL, FURA, MATA, INDO, and derivatives thereof.

23. The method of claim 19, wherein the chilled platelets are treated with a second agent for inhibiting actin polymerization.

24. The method of claim 19, wherein the second agent is a cytochalasin.

25. The method of claim 24, wherein the cytochalasin is selected from the group consisting of: cytochalasin B and dihydro-cytochalasin B.

26. The method of claim 19, wherein the chilled platelets are treated with a first agent for inhibiting actin filament severing and a second agent for inhibiting actin polymerization.

27. The method of claim 26, wherein the first agent is an intracellular calcium chelator and the second agent is a cytochalasin.

28. The method of claim 27, wherein the intracellular calcium chelator is selected from the group consisting of QUIN, STIL, FURA, MATA, INDO, and derivatives thereof.

29. The method of claim 27, wherein the cytochalasin is selected from the group consisting of cytochalasin B and dihydro-cytochalasin B.

30. The method of claim 13, further comprising the step of separating the platelet antagonist-treated platelet from the platelet antagonist that has not bound to a chilled platelet.

31. The method of claim 13 or 30, further comprising the step of contacting the chilled platelet or the platelet antagonist-treated platelet with a liver macrophage receptor antagonist.

32. The method of claim 19, wherein the liver macrophage receptor antagonist binds to a liver macrophage receptor selected from the group of liver macrophage receptors provided in Table 1.

33. The method of claim 32, wherein the liver macrophage receptor antagonist binds to a liver macrophage receptor that is αMβ2.

34. The method of claims 13, 30, or 31, further comprising the step of administering the platelet antagonist-treated platelet to a subject.

35. A method for forming a medicament, comprising:
placing a plurality of chilled platelets and one or more platelet clearance antagonists in a pharmaceutically acceptable carrier.

36. The method of claim 35, wherein the platelet clearance antagonist is a platelet antagonist.

37. The method of claim 35, wherein the platelet clearance antagonist is a liver macrophage receptor antagonist.

38. A composition comprising:
A plurality of platelets; and
One or more platelet clearance antagonists.

39. The composition of claim 38, wherein the composition comprises a first platelet clearance antagonist that is a platelet antagonist and a second platelet clearance antagonist that is a liver macrophage

40. The composition of claim 38, wherein the platelet clearance antagonist is a platelet antagonist.

41. The composition of claim 40, wherein the platelet clearance antagonist is a liver macrophage receptor antagonist.

42. The composition of claim 38, wherein the composition is contained in a platelet bag.

43. The composition of claim 38, wherein the platelet clearance antagonist is a platelet antagonist that selectively binds to a platelet ligand identified in Table 1.

44. The composition of claim 43, wherein the platelet antagonist selectively binds to vWfR or a subunit thereof.

45. The composition of claim 38, wherein the platelet clearance antagonist is a liver macrophage receptor antagonist that selectively binds to a liver macrophage receptor identified in Table 1.

46. The composition of claim 45, wherein the liver macrophage receptor antagonist selectively binds to αMβ2.

47. The composition of claim 38, further comprising a pharmaceutically acceptable carrier.

48. A method for increasing platelet circulatory time, comprising:
Administering to a subject in need of such treatment, a composition comprising:
One or more platelet clearance antagonists in an amount effective to increase platelet circulatory time in the subject.

49. The method of claim 48, wherein the composition comprises a first platelet clearance antagonist that is a platelet antagonist and a second platelet clearance antagonist that is a liver macrophage

50. The method of claim 48, wherein the platelet clearance antagonist is a platelet antagonist.

51. The method of claim 50, wherein the platelet clearance antagonist is a liver macrophage receptor antagonist.

52. The method of claim 48, wherein the composition is contained in a platelet bag.

53. The method of claim 48, wherein the platelet clearance antagonist is a platelet antagonist that selectively binds to a platelet ligand identified in Table 1.

54. The method of claim 53, wherein the platelet antagonist selectively binds to vWfR or a subunit thereof.

55. The method of claim 48, wherein the platelet clearance antagonist is a liver macrophage receptor antagonist that selectively binds to a liver macrophage receptor identified in Table 1.

56. The method of claim 55, wherein the liver macrophage receptor antagonist selectively binds to αMβ2.

57. A method of treating a subject in need of platelets, comprising:
Administering to the subject,
(1) a first composition comprising:
A plurality of chilled platelets; and
One or more platelet clearance antagonists; or
(2) a second composition comprising:
a plurality of platelet-antagonist-treated platelets,
Wherein the first composition or the second composition is administered in an amount effective to treat the subject.

58. The method of claim 57, wherein the administering the second composition further includes administering a liver macrophage receptor antagonist to the subject.

59. The composition of claim 57, wherein the composition comprises a first platelet clearance antagonist that is a platelet antagonist and a second platelet clearance antagonist that is a liver macrophage receptor antagonist.

60. The composition of claim 57, wherein the platelet clearance antagonist is a platelet antagonist.

61. The composition of claim 60, wherein the platelet clearance antagonist is a liver macrophage receptor antagonist.

62. The composition of claim 57, wherein the composition is contained in a platelet bag.

63. The composition of claim 57, wherein the platelet clearance antagonist is a platelet antagonist that selectively binds to a platelet ligand identified in Table 1.

64. The composition of claim 63, wherein the platelet antagonist selectively binds to vWfR or a subunit thereof.

65. The composition of claim 57, wherein the platelet clearance antagonist is a liver macrophage receptor antagonist that selectively binds to a liver macrophage receptor identified in Table 1.

66. The composition of claim 65, wherein the liver macrophage receptor antagonist selectively binds to αMβ2.

67. A method for identifying a platelet lesion cleavage agent, comprising:
Contacting a chilled platelet with a liver macrophage in the presence and in the absence of a test cleavage agent; and
Detecting binding of the chilled platelet to the liver macrophage,
Wherein an increase in the binding in the presence of the test cleavage agent relative to the binding in the absence of the test cleavage agent indicates that the test molecule is a platelet lesion cleavage agent.

68. The method of claim 67, wherein test cleavage agent is selected from the group consisting of enzymes that cleave carbohydrates.

69. The method of claim 68, wherein the enzyme is a galactosidase, a glucosidase, a mannosidase.

70. The method of claim 67, wherein detecting binding of the chilled platelet to the liver macrophage comprises detecting phagocytosis of the chilled platelet by the liver macrophage.

71. The method of claim 67, wherein the platelet lesion comprises surface expression of vWfR or a subunit thereof by the chilled platelet.

72. The method of claim 67, further comprising the step of contacting the platelet lesion cleavage agent with a chilled platelet under conditions to permit the platelet lesion cleavage agent to cleave a platelet lesion on a chilled platelet.

73. A platelet prepared in accordance with the method of claim 72.
